(19) United States
(12) Patent Application Publication
(10) Pub. No.: US 2022/0415442 A1
(43) Pub. Date: Dec. 29, 2022

(54) SIGNAL-TO-NOISE-RATIO METRIC FOR DETERMINING NUCLEOTIDE-BASE CALLS AND BASE-CALL QUALITY

(71) Applicant: Illumina Software, Inc., San Diego, CA (US)

(72) Inventors: Eric Jon Ojard, San Francisco, CA (US); Nitin Udpa, San Diego, CA (US); Abde Ali Kagalwalla, San Diego, CA (US); John S Vieceli, San Diego, CA (US); Rami Mehio, San Diego, CA (US)

(21) Appl. No.: 17/805,138

(22) Filed: Jun. 2, 2022

Related U.S. Application Data
(60) Provisional application No. 63/216,401, filed on Jun. 29, 2021.

(51) Int. Cl. G16B 30/10; C12Q 1/6869; G16B 40/10

(52) U.S. Cl. CPC G16B 30/10; C12Q 1/6869; G16B 40/10

(57) ABSTRACT

This disclosure describes methods, non-transitory computer readable media, and systems that can generate signal-to-noise-ratio metrics for clusters of oligonucleotides to which tagged nucleotide bases are added and utilize the signal-to-noise-ratio metrics to generate nucleotide-base calls and determine base-call quality. For example, the disclosed systems can generate the signal-to-noise-ratio metrics using scaling factors and noise levels associated with light signals detected from the clusters of oligonucleotides. The disclosed systems can utilize the signal-to-noise-ratio metrics to generate intensity-value boundaries for generating nucleotide-base-calls for the signals in accordance with one or more base-call-distribution models. Additionally, the disclosed systems can utilize a threshold to filter out signals detected from the clusters of oligonucleotides that have low signal-to-noise-ratio metrics. The disclosed systems can further utilize the signal-to-noise-ratio metrics to generate quality metrics for generated nucleotide-base calls.

### CROSS-REFERENCE TO RELATED APPLICATIONS

This application claims the benefit of, and priority to, U.S. Provisional Application No. 63/216,401, entitled “SIGNAL-TO-NOISE-RATIO METRIC FOR DETERMINING NUCLEOTIDE-BASE CALLS AND BASE-CALL QUALITY,” filed Jun. 29, 2021, the contents of which are hereby incorporated by reference in their entirety.

### BACKGROUND

In recent years, biotechnology firms and research institutions have improved hardware and software platforms used for determining a sequence of nucleotide bases (also referred to as “nucleobases”) in a sample of nucleic acid. For instance, some existing nucleic-acid-sequencing platforms determine individual nucleotide bases of nucleic-acid sequences by using conventional Sanger sequencing or by using sequencing-by-synthesis (SBS). When using SBS, existing platforms can monitor thousands, tens of thousands, or more nucleic-acid polymers being synthesized in parallel to detect more accurate nucleotide-base calls. For instance, a camera in SBS platforms can capture images of irradiated fluorescent tags from nucleotide bases incorporated into such synthesized nucleic-acid sequences (often grouped into clusters). After capturing the images, existing SBS platforms send image data to a computing device with sequencing-data-analysis software to determine a nucleotide-base sequence for a nucleic-acid polymer. The sequencing-data-analysis software can determine the nucleotide bases that were detected in a given image based on the light signal captured in the image data. By iteratively incorporating nucleotide bases into the oligonucleotides and capturing images of the emitted light signals in various sequencing cycles, the SBS platforms can determine the sequence of nucleotide bases present in the samples of nucleic acid.

Despite these recent advances, existing sequencing platforms typically suffer from technical limitations that impede the accuracy and flexibility of those platforms. In particular, rigid intensity-value-boundary models often hinder such sequencing platforms from interpreting the light signals captured in the image data to make correct nucleotide-base calls. Further, flawed base-call-quality models and filtering models tend to restrict the ability of such platforms to determine the accuracy of determined nucleotide-base calls.

Indeed, intensity-value-boundary models of existing sequencing platforms often result in inaccuracies when interpreting the light signals emitted from irradiated fluorescent tags of nucleotide bases to classify those nucleotide bases when making nucleotide-base calls. For example, some existing platforms generate nucleotide-base calls using decision boundaries that map intensity values (e.g., wavelength and/or brightness values) associated with light signals to corresponding nucleotide bases. These platforms, however, may use decision boundaries that are inappropriate (e.g., do not accurately map intensity values to nucleotide bases) for a given light signal, leading to an inaccurate nucleotide-base call. Such inaccurate calls are often caused by the rigid application by some existing platforms of the same set of decision boundaries for all light signals. Indeed, existing sequencing platforms may use a single model (e.g., a single Gaussian mixture model) to generate the decision boundaries used for all detected light signals. Different light signals, however, may have varying factors—such as varying levels of signal purity—that affect the associated intensity values. By failing to account for these factors, existing platforms fail to flexibly tailor the decision boundaries to the characteristics of the light signals.

Some existing sequencing platforms attempt to circumvent the inaccuracies of generating nucleotide-base calls by filtering out problematic clusters of nucleic-acid polymers (e.g., excluding corresponding nucleotide-base calls from the resulting base-call data). For example, existing platforms may filter out clusters of nucleic-acid polymers using a chastity filter, which analyzes the chastity values of the corresponding light signals. The chastity value can be determined as the ratio of the distance between the intensity associated with a light signal and the nearest nucleotide-base centroid to the distance between the intensity and another centroid (e.g., the second nearest centroid).

Existing platforms may filter out nucleotide-base calls for a cluster if its chastity values fail to satisfy a threshold (e.g., multiple times within a first set of sequencing cycles), indicating that the emitted light signals are of poor quality and unreliable (e.g., the corresponding nucleotide-base calls may be inaccurate). Clusters, however, may become more problematic as sequencing progresses. Indeed, the poor quality of a cluster that satisfies the chastity filter in early sequencing cycles may surface in later sequencing cycles. By using the chastity filter, many existing platforms fail to properly identify these problematic clusters. Thus, such platforms tend to generate unreliable nucleotide-base calls based on the poor light signals emitted from these clusters and include those nucleotide-base calls in the base-call data.

In addition to problems with generating accurate nucleotide-base calls and filtering out nucleic-acid polymers that emit unreliable light signals, existing sequencing platforms are often inaccurate when determining the quality of a given nucleotide-base call. For example, many existing platforms determine a metric, such as the Phred quality score, that estimates the likelihood of error of a nucleotide-base call. The models used to determine this quality score, however, leave many features associated with the nucleotide-base call (e.g., associated with the corresponding light signal) unconsidered, even where such features contribute significantly to the quality of the nucleotide-base call. Thus, existing platforms often fail to accurately estimate the quality of a nucleotide-base call.

Further, as previously mentioned, existing platforms fail to tailor the decision boundaries used for generating a nucleotide-base call to the characteristics of light signals. In many instances, quality estimations are inherently tied to the decision boundaries used in generating nucleotide-base calls. Thus, using decision boundaries that fail to accurately map the intensity values of light signals to nucleotide bases can also lead to inaccurate estimations of the quality of the resulting nucleotide-base calls.

### SUMMARY

This disclosure describes embodiments of methods, non-transitory computer-readable media, and systems that determine signal-to-noise-ratio metrics for light signals emitted from fluorescent tags of nucleotide bases and use such signal-to-noise-ratio metrics to determine more accurate and flexible base calls. For example, the disclosed systems can determine a separate signal-to-noise-ratio metric for various clusters of oligonucleotides to which tagged nucleotide bases are added. The disclosed systems can utilize the intensity values associated with the light signal emitted from a cluster to determine its corresponding signal-to-noise-ratio metric. For instance, the disclosed systems determine a signal-to-noise-ratio metric for labeled nucleotide bases in a cluster of oligonucleotides based on a scaling factor and a noise level for the cluster's light signal. In some cases, the disclosed systems update the signal-to-noise-ratio metric after every sequencing cycle.

The disclosed systems can utilize such signal-to-noise-ratio metrics associated with the clusters for a variety of base-calling applications described further below. For example, the disclosed systems can use such signal-to-noise-ratio metrics to generate intensity-value boundaries for differentiating signals corresponding to different nucleotide bases according to a base-call-distribution model (e.g., segmented Gaussian mixture model), filter out clusters of poor quality, and/or determine a quality score for nucleotide-base calls. By utilizing such a signal-to-noise-ratio metric, the disclosed systems flexibly tailor the decision boundaries between different nucleotide clouds used for determining nucleotide-base calls to the characteristics of detected light signals, allowing for more accurate base calling. Further, the disclosed systems can utilize the signal-to-noise-ratio metrics to more accurately filter poor-quality wells and more accurately determine the quality score of a given nucleotide-base call.

Additional features and advantages of one or more embodiments of the present disclosure are outlined in the following description.

### BRIEF DESCRIPTION OF THE DRAWINGS

The detailed description refers to the drawings briefly described below.

FIG. 1 illustrates a block diagram of a sequencing system including a signal-to-noise-aware base calling system in accordance with one or more embodiments.

FIG. 2 illustrates an overview diagram of the signal-to-noise-aware base calling system generating and utilizing a signal-to-noise-ratio metric in accordance with one or more embodiments.

FIG. 3 illustrates a diagram for determining a signal-to-noise-ratio metric in accordance with one or more embodiments.

FIG. 4 illustrates a block diagram of utilizing signal-to-noise-ratio metrics for distribution-model segmentation in accordance with one or more embodiments.

FIG. 5 illustrates a block diagram for utilizing a signal-to-noise-ratio metric of a signal to filter nucleotide-base calls in accordance with one or more embodiments.

FIG. 6 illustrates a block diagram for generating a quality metric for a nucleotide-base call in accordance with one or more embodiments.

FIG. 7 illustrates a graph reflecting research results regarding the effectiveness of the signal-to-noise-aware base calling system in accordance with one or more embodiments.

FIGS. 8A-8B illustrate graphs reflecting additional research results regarding the effectiveness of the signal-to-noise-aware base calling system in accordance with one or more embodiments.

FIG. 9 illustrates a flowchart of a series of acts for generating a quality metric for a nucleotide-base call using a signal-to-noise-ratio metric in accordance with one or more embodiments.

FIG. 10 illustrates a flowchart of a series of acts for filtering a nucleotide-base call corresponding to a signal using a signal-to-noise-ratio metric in accordance with one or more embodiments.

FIG. 11 illustrates a flowchart of a series of acts for generating intensity-value boundaries for signal-to-noise ranges using signal-to-noise-ratio metrics in accordance with one or more embodiments.

FIG. 12 illustrates a block diagram of an example computing device for implementing one or more embodiments of the present disclosure.

### DETAILED DESCRIPTION

This disclosure describes one or more embodiments of a signal-to-noise-aware base calling system that utilizes a signal-to-noise-ratio metric for determining nucleotide-base calls, measuring the quality of the nucleotide-base calls, and filtering out poor-quality wells. In particular, in some implementations, the signal-to-noise-aware base calling system determines a signal-to-noise-ratio metric for a section of a nucleotide-sample slide (e.g., a well of a patterned flow cell or a subsection of a non-patterned flow cell) containing a cluster of oligonucleotides. For example, the signal-to-noise-aware base calling system can determine the signal-to-noise-ratio metric based on a scaling factor and a noise level corresponding to intensity values of the light signal emitted by the cluster.

The signal-to-noise-aware base calling system can utilize such signal-to-noise-ratio metrics to determine better quality or more accurate nucleobase calls through a variety of applications. For instance, in some cases, the signal-to-noise-aware base calling system utilizes signal-to-noise-ratio metrics to generate intensity-value boundaries for differentiating signals corresponding to different nucleotide bases in accordance with one or more base-call-distribution models (e.g., a segmented Gaussian mixture model). In some instances, the signal-to-noise-aware base calling system uses or establishes a signal-to-noise threshold and filters nucleotide-base calls associated with the section of the nucleotide-sample slide out of the sequencing data if the signal-to-noise-ratio metric fails to satisfy the threshold. In some embodiments, the signal-to-noise-aware base calling system utilizes the signal-to-noise-ratio metric as an input to a model (e.g., a Phred algorithm) that estimates the quality of a nucleotide-base call generated for the section of the nucleotide-sample slide.

As just mentioned, in one or more embodiments, the signal-to-noise-aware base calling system determines a signal-to-noise-ratio metric for a section of a nucleotide-sample slide. In one or more embodiments, the signal-to-noise-ratio metric is specific to that section of the nucleotide-sample slide, and the signal-to-noise-aware base calling system determines other signal-to-noise-ratio metrics for other sections of the nucleotide-sample slide. In one or more embodiments, the signal-to-noise-aware base calling system updates the signal-to-noise-ratio metric for a section of the nucleotide-sample slide with each sequencing cycle.

As suggested above, in one or more embodiments, the signal-to-noise-aware base calling system determines the signal-to-noise-ratio metric for a section of a nucleotide-sample slide based on the intensity values of a signal (e.g., light signal) detected from the section of the nucleotide-sample slide. For example, the signal-to-noise-aware base calling system can determine a scaling factor for the detected signal. In some cases, the signal-to-noise-aware base calling system determines the scaling factor using a least squares algorithm based on the intensity values of the signal. The signal-to-noise-aware base calling system can further determine a noise level corresponding to the detected signal. For instance, in some embodiments, the signal-to-noise-aware base calling system determines the noise level based on corrected intensity values for the signal. The signal-to-noise-aware base calling system can determine the signal-to-noise-ratio metric based on both the scaling factor and the noise level.

As further mentioned above, in some implementations, the signal-to-noise-aware base calling system utilizes signal-to-noise-ratio metrics to generate intensity-value boundaries for differentiating signals corresponding to different nucleotide bases. To illustrate, in certain cases, the signal-to-noise-aware base calling system generates signal-to-noise-ratio metrics for a plurality of sections of the nucleotide-sample slide (e.g., based on the signals detected during a sequencing cycle). The signal-to-noise-aware base calling system can determine signal-to-noise-ratio ranges for the determined signal-to-noise-ratio metrics and fit a base-call-distribution model to the nucleotide-sample slide sections associated with each signal-to-noise-ratio range. The signal-to-noise-aware base calling system can then generate a nucleotide-base call for a section of the nucleotide-sample slide in accordance with the base-call-distribution model of the signal-to-noise-ratio range that encompasses the signal-to-noise-ratio metric for that section of the nucleotide-sample slide.

Additionally, as mentioned above, in one or more embodiments, the signal-to-noise-aware base calling system utilizes the signal-to-noise-ratio metric of a nucleotide-sample slide section in determining whether to filter out corresponding nucleotide-base calls from the nucleotide-base-call data (e.g., sequencing data) that results from the sequencing. Indeed, in some implementations, the signal-to-noise-aware base calling system establishes a signal-to-noise-ratio threshold. Upon determining that the signal-to-noise-ratio metric satisfies the signal-to-noise-ratio threshold, the signal-to-noise-aware base calling system can determine and include nucleotide-base calls for the section of the nucleotide-sample slide within the nucleotide-base-call data. If the signal-to-noise-ratio metric fails to satisfy the signal-to-noise-ratio threshold, the signal-to-noise-aware base calling system can exclude nucleotide-base calls for the section of the nucleotide-sample slide from the nucleotide-base-call data.

In addition (or in the alternative) to generating intensity-value boundaries or filtering, in one or more embodiments, the signal-to-noise-aware base calling system utilizes the signal-to-noise-ratio metric of a section of a nucleotide-sample slide to estimate the quality of a nucleotide-base call generated for the section of the nucleotide-sample slide. For instance, in some cases, the signal-to-noise-aware base calling system provides the signal-to-noise-ratio metric as an input to a base-call-quality model (e.g., a Phred algorithm). The signal-to-noise-aware base calling system can utilize the base-call-quality model to generate a quality metric that estimates an error of the nucleotide-base call based on the signal-to-noise-ratio metric. In some implementations, the signal-to-noise-aware base calling system provides the signal-to-noise-ratio metric as one of many inputs (e.g., together with a chastity value) to the base-call-quality model.

The signal-to-noise-aware base calling system provides several advantages over conventional sequencing platforms. For example, as an initial matter, the signal-to-noise-aware base calling system introduces a new computational model for determining a signal-to-noise-ratio metric for light signals emitted by fluorescent tags and captured by a camera. In particular, the disclosed computational model determines the signal-to-nose-ratio metric corresponding to a light signal by disaggregating and relating the purity of the light signal to the noise associated with the light wavelength or intensity emitted by the fluorescent tags. For example, as described above and below, the computational model can deconstruct a detected light signal into a scaling factor and a noise level and determine the signal-to-noise-ratio metric based on these values. By doing so, the computational model can more accurately distinguish between a light signal corresponding to a nucleotide-base call and noise. The human mind cannot detect light signals emitted from labeled nucleotide bases, much less separate the light signal from associated noise. Accordingly, by determining signal-to-noise-ratio metrics, the new computational model provides functionality that was previously unavailable to sequencing platforms.

By utilizing the signal-to-noise-ratio metric, the signal-to-noise-aware base calling system improves nucleotide-base calling. For example, as discussed above, the signal-to-noise-aware base calling system fits the base-call-distribution models used for generating nucleotide-base calls to various signal-to-noise-ratio ranges. These base-call-distribution models provide intensity-value boundaries (e.g., decision boundaries) upon which nucleotide-base calls are based. Thus, the signal-to-noise-aware base calling system flexibly tailors the intensity-value boundaries to the various levels of signal purity associated with the signals detected from sections of the nucleotide-sample slide. As further demonstrated by the results described below, the signal-to-noise-aware base calling system improves nucleotide-base calls for sections of the nucleotide-sample slide using intensity-value boundaries that are appropriate for their emitted signals, resulting in more accurate nucleotide-base calls.

By utilizing the signal-to-noise-ratio metric, the signal-to-noise-aware base calling system also filters out poor-quality base calls for sections of a nucleotide-sample slide. In particular, the signal-to-noise-aware base calling system more accurately identifies sections of the nucleotide-sample slide that are emitting poor signals. Indeed, the signal-to-noise-aware base calling system can identify those sections of the nucleotide-sample slide that would otherwise pass a chastity filter implemented by conventional sequencing platforms only to surface their errors in later sequencing cycles. By improving the filtering process, the signal-to-noise-aware base calling system generates more accurate, more reliable nucleotide-base-call data.

In addition to improved nucleotide-base calls and improved filtering, the signal-to-noise-aware base calling system more accurately determines nucleotide-base-call quality than conventional sequencing platforms. Indeed, by utilizing the signal-to-noise-ratio metric, the signal-to-noise-aware base calling system can more accurately estimate the quality of a nucleotide-base call. For example, as mentioned above, the signal-to-noise-aware base calling system can provide the signal-to-noise-ratio metric of a section of a nucleotide-sample slide as input to a base-call-quality model (e.g., Phred model). Accordingly, the signal-to-noise-aware base calling system utilizes a novel and improved (and sometimes additional) indicator of nucleotide-base-call quality when compared to conventional sequencing platforms, allowing for more accurate quality estimates. Further, by using intensity-value boundaries that are tailored to the characteristics of detected light signals, the quality estimations tied to those intensity-value boundaries are also tailored to the characteristics of the light signals.

As illustrated by the foregoing discussion, the present disclosure utilizes a variety of terms to describe features and advantages of the signal-to-noise-aware base calling system. Additional detail is now provided regarding the meaning of such terms. For example, as used herein, the term “nucleotide-sample slide” refers to a plate or slide comprising oligonucleotides for sequencing nucleotide segments for samples. In particular, a nucleotide-sample slide can refer to a slide containing fluidic channels through which reagents and buffers can travel as part of sequencing. For example, in one or more embodiments, a nucleotide-sample slide includes a flow cell (e.g., a patterned flow cell or non-patterned flow cell) comprising small fluidic channels and short oligonucleotides complementary to adaptor sequences.

Relatedly, as used herein, the term “section of a nucleotide-sample slide” (or “nucleotide-sample slide section”) refers to an area that is part of a nucleotide-sample slide. In particular, a section of a nucleotide-sample slide can refer to a discrete portion of a nucleotide-sample slide that differs from other portions of the nucleotide-sample slide. For instance, a section of a nucleotide sample slide can include a well (e.g., a nanowell) of a patterned flow cell or a discrete subsection of a non-pattered flow cell (e.g., a subsection corresponding to a cluster). In some cases, a section of a nucleotide sample slide includes a tile or a sub-tile having clusters of the same or similar oligonucleotide growing in parallel.

Additionally, as used herein, the term “labeled nucleotide base” refers to a nucleotide base having a fluorescent or light-based indicator of the classification of the nucleotide base. In particular, a labeled nucleotide base can refer to a nucleotide base that incorporates a fluorescent or light-based indicator to identify the type of base (e.g., adenine, cytosine, thymine, or guanine). For example, in one or more embodiments, a labeled nucleotide base includes a nucleotide base having a fluorescent tag that emits a signal that identifies the base type.

Further, as used herein the term “signal” refers to a signal emitted, reflected, or otherwise communicated from a labeled nucleotide base or a group of labeled nucleotide bases (e.g., labeled nucleotide bases added to a cluster of oligonucleotides). In particular, a signal can refer to a signal indicating the type of base. For example, a signal can include a light signal emitted or reflected from a fluorescent tag of a nucleotide base or fluorescent tags of multiple nucleotide bases incorporated into oligonucleotides. In some implementations, the signal-to-noise-aware base calling system triggers the signal through an external stimulus, such as a laser or other light source. In some cases, the signal-to-noise-aware base calling system triggers the signal through some internal stimuli. Further, in some embodiments, the signal-to-noise-aware base calling system observes the signal using a filter applied when capturing an image of the nucleotide-sample slide (e.g., section of the nucleotide-sample slide). As suggested above, in certain instances, a signal includes an aggregate of the signals provided by each labeled nucleotide base added to individual oligonucleotides in a cluster of oligonucleotides.

As used herein, the term “intensity value” refers to a value indicating a characteristic or attribute of a signal emitted, reflected, or otherwise communicated from a labeled nucleotide base or a group of labeled nucleotide bases from a cluster of oligonucleotides. In particular, an intensity value can refer to a value associated with a color intensity (e.g., wavelength) or a light intensity (e.g., brightness). In some cases, the signal-to-noise-aware base calling system captures several images of a cluster of oligonucleotides with labeled nucleotide bases using different filters (or intensity channels). Thus, an intensity value of a signal can correspond to the intensity of the signal as observed through a particular filter.

Additionally, as used herein, the term “signal-to-noise-ratio metric” refers to a measure of a target signal compared to a level or content of noise. In particular, a signal-to-noise-ratio metric can refer to the strength of a light signal that is detected from labeled nucleotide bases compared to associated noise. For example, in some implementations, a signal-to-noise-ratio metric includes a ratio of a scaling factor associated with a signal compared to the corresponding noise level. As used herein, the term “scaling factor” refers to a coefficient or value that indicates brightness. In particular, as used herein, the term scaling factor can refer to a value that accounts for scale variation (e.g., amplitude/brightness variation) in an inter-cluster intensity profile variation (which relates to the difference in scale and shifts from an origin of a multi-dimensional space of the intensity profiles of clusters in a cluster population). In one or more embodiments, the signal-to-noise-aware base calling system equates the scaling factor determined for a light signal to the light signal itself (e.g., the signal purity without the addition of noise). Further, as used herein, the term “noise level” refers to a value indicating the noise associated with a signal. Indeed, in some cases, a noise level includes a value indicating noise that comprises signal variation that leads to (or reflects) a distribution in an observed population. The signal variation can come from chemical or physical properties of components or contents of a nucleotide-sample slide or of a sequencing device, such as signal variation attributable to oligonucleotide length, phasing or pre-phasing, or a position of a cluster of oligonucleotides with respect to a camera or other sensor's field of view. In one or more embodiments, as will be discussed in more detail below, the signal-to-noise-aware base calling system determines the scaling factor and the noise level using one or more intensity values of the signal. As used herein, the term “signal-to-noise-ratio range” refers to a range of signal-to-noise-ratio metrics. In other words, in some implementations, the signal-to-noise-aware base calling system establishes one or more signal-to-noise-ratio ranges and determines whether the signal-to-noise-ratio metric of a signal falls within a particular signal-to-noise-ratio range.

Further, as used herein, the term “signal-to-noise-ratio threshold” refers to a threshold value established for filtering out a cluster of oligonucleotides (e.g., nucleotide-base calls associated with the cluster of oligonucleotides) based on the signal-to-noise-ratio metric. For example, in some implementations, the signal-to-noise-aware base calling system determines a signal-to-noise-ratio threshold as a signal-to-noise-ratio value that must be satisfied (e.g., met or exceeded) by a signal from labeled nucleotide bases corresponding to a cluster of oligonucleotides to have nucleotide-base calls for the cluster to be included in the resulting nucleotide-base-call data.

As used herein, the term “nucleotide-base call” refers to an assignment or determination of a particular nucleotide base to add to or incorporate within an oligonucleotide for a sequencing cycle. In particular, a nucleotide-base call indicates an assignment or a determination of the type of nucleotide that has been incorporated within an oligonucleotide on a nucleotide-sample slide. In some cases, a nucleotide-base call includes an assignment or determination of a nucleotide base to intensity values resulting from nucleotides added to an oligonucleotide in a section of a nucleotide-sample slide. Alternatively, a nucleotide-base call includes an assignment or determination of a nucleotide base to chromatogram peaks or electrical current changes resulting from nucleotides passing through a nanopore of a nucleotide-sample slide. By using nucleotide-base calls, a sequencing system determines a sequence of a nucleic-acid polymer. For example, a single nucleotide-base call can comprise an adenine call, a cytosine call, a guanine call, or a thymine call.

Additionally, as used herein, the term “sequencing cycle” (or “cycle”) refers to an iteration of adding or incorporating a nucleotide base to an oligonucleotide or an iteration of adding or incorporating nucleotide bases to oligonucleotides in parallel. In particular, a cycle can include an iteration of taking an analyzing one or more images with data indicating individual nucleotide bases added or incorporated into an oligonucleotide or to oligonucleotides in parallel. Accordingly, cycles can be repeated as part of sequencing a nucleic-acid polymer. For example, in one or more embodiments, each sequencing cycle involves either single reads in which DNA or RNA strands are read in only a single direction or paired-end reads in which DNA or RNA strands are read from both ends. Further, in certain cases, each sequencing cycle involves a camera taking an image of the nucleotide-sample slide or multiple sections of the nucleotide-sample slide to generate image data for determining a particular nucleotide base added or incorporated into particular oligonucleotides. Following the image capture stage, a sequencing system can remove certain fluorescent labels from incorporated nucleotide bases and perform another sequencing cycle until the nucleic-acid polymer has been completely sequenced. In one or more embodiments, a sequencing cycle includes a cycle within a Sequencing By Synthesis (SBS) run.

Additionally, as used herein, the term “nucleotide-base-call data” refers to a digital file, image data, or other digital information indicating individual nucleotide bases or the sequence of nucleotide bases for a nucleic-acid polymer. In particular, nucleotide-base-call data can include intensity values (e.g., color or light intensity values for individual clusters) from images taken by a camera of a nucleotide-sample slide or other data that indicate individual nucleotide bases or the sequence of nucleotide bases for a nucleic-acid polymer. In addition, or in the alternative to intensity values, the nucleotide-base-call data may include chromatogram peaks or electrical current changes indicating individual nucleobases in a sequence. Additionally, in some embodiments, nucleotide-base-call data includes individual nucleotide-base calls identifying the individual nucleotide bases (e.g., A, T, C, or G). For example, nucleotide-base-call data can comprise data for nucleotide-base calls in a sequence for a nucleic-acid polymer, the number of nucleotide-base calls corresponding to a particular base (e.g., adenine, cytosine, thymine, or guanine), as organized in a digital file, such as a Binary Base Call (BCL) file. Further, nucleotide-base call data can include error/accuracy information, such as a quality metric associated with each nucleotide-base call. In some embodiments, nucleotide-base-call data comprises information from a sequencing device that utilizes sequencing by synthesis (SBS).

As used herein, the term “quality metric” refers to a specific score or other measurement indicating the accuracy of nucleotide-base calls for a sequencing cycle. In particular, a quality metric comprises a value indicating the likelihood that one or more predicted nucleotide-base calls contain errors. For example, in certain implementations, a quality metric can comprise a Q score (e.g., a Phred quality score) predicting the error probability of any given nucleotide-base call within a sequencing cycle.

As used herein, the term “base-call-quality model” refers to a computer model or algorithm that generates a quality metric for a nucleotide-base call. For example, a base-call-quality model can refer to a computer algorithm that analyzes characteristics of a signal and/or the corresponding cluster or labeled nucleotide bases and generates a quality metric for the nucleotide-base call based on the analysis. To illustrate, in some implementations, the base-call-quality model includes a computer algorithm that generates a Phred quality score.

Additionally, as used herein, the term “intensity-value boundaries” refers to decision boundaries used in generating a nucleotide-base call for a signal. In particular, intensity-value boundaries can refer to decision boundaries that classify a nucleotide base (e.g., as A, T, C, or G) based on one or more intensity values of the signal. To illustrate, intensity-value boundaries can define or otherwise indicate the boundaries of a nucleotide cloud corresponding to each of the nucleotide bases. In some implementations, intensity-value boundaries do not mark the limits at which a signal is classified as a nucleotide base, but rather a point at which the signal can be classified as the nucleotide base with a particular level of accuracy.

As used herein, the term “base-call-distribution model” refers to a computer model or algorithm that generates intensity-value boundaries. For example, in some implementations, a base-call-distribution model includes, but is not limited to, a Gaussian distribution model, a uniform distribution model, a Bernoulli distribution model, a binomial distribution model, or a Poisson distribution model. As used herein, the term “centroid” refers to the center of a nucleotide cloud defined or otherwise indicated by one or more intensity-value boundaries. Further, as used herein, the term “centroid intensity value” refers to an intensity value associated with a centroid. In particular, a centroid intensity value indicates an intensity value that corresponds to the center of a nucleotide cloud.

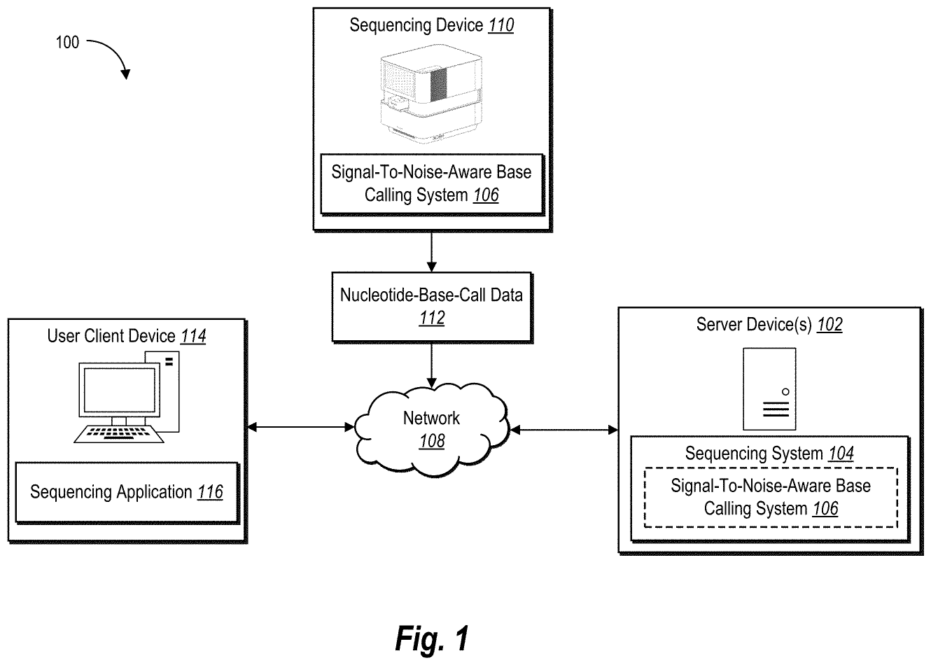

The following paragraphs describe the signal-to-noise-aware base calling system with respect to illustrative figures that portray example embodiments and implementations. For example, FIG. 1 illustrates a schematic diagram of a system environment (or “environment”) 100 in which a signal-to-noise-aware base calling system 106 operates in accordance with one or more embodiments. As illustrated, the environment 100 includes one or more server device(s) 102 connected to a sequencing device 110 and a user client device 114 via network 108. While FIG. 1 shows an embodiment of the signal-to-noise-aware base calling system 106, this disclosure describes alternative embodiments and configurations below.

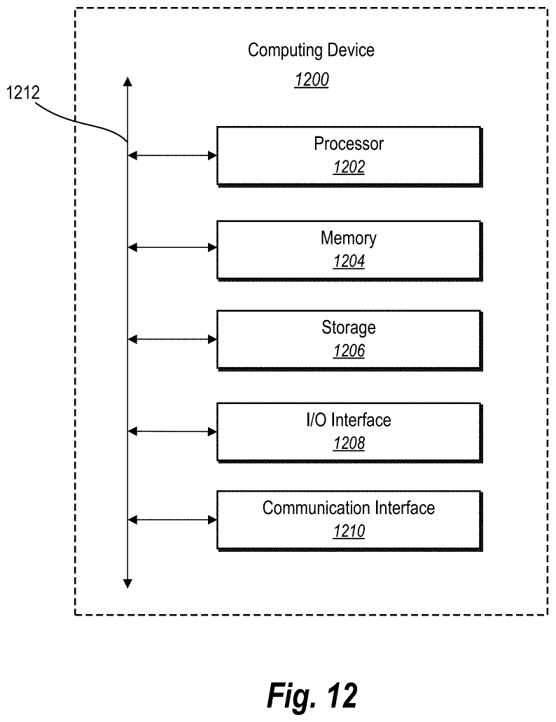

As shown in FIG. 1, the server device(s) 102, the sequencing device 110 and the user client device 114 are connected via the network 108. Accordingly, each of the components of the environment 100 can communicate via the network 108. The network 108 comprises any suitable network over which computing devices can communicate. Example networks are discussed in additional detail below with respect to FIG. 12.

As indicated by FIG. 1, the sequencing device 110 comprises a device for sequencing a nucleic-acid polymer. In some embodiments, the sequencing device 110 analyzes nucleic-acid segments or oligonucleotides extracted from samples to generate data utilizing computer implemented methods and systems either directly or indirectly on the sequencing device 110. More particularly, the sequencing device 110 receives and analyzes, within nucleotide-sample slides (e.g., flow cells), nucleic-acid sequences extracted from samples. In one or more embodiments, the sequencing device 110 utilizes SBS to sequence nucleic-acid polymers. In addition, or in the alternative to communicating across the network 108, in some embodiments, the sequencing device 110 bypasses the network 108 and communicates directly with the server device(s) 102 and/or the user client device 114.

As just mentioned, and as illustrated in FIG. 1, the signal-to-noise-aware base calling system 106 can generate or at least contribute to generating nucleotide-base-call data 112. In particular, in some embodiments, the signal-to-noise-aware base calling system 106 generates the nucleotide-base-call data 112 utilizing signal-to-noise-ratio metrics. To illustrate, in some cases, the signal-to-noise-aware base calling system 106 determines a signal-to-noise-ratio metric for sections of a nucleotide-sample slide (e.g., for the signals detected from those sections) during each sequencing cycle. The signal-to-noise-aware base calling system 106 can utilize the signal-to-noise-ratio metric for each section to generate a nucleotide-base call corresponding to the signal detected from that section. The signal-to-noise-aware base calling system 106 can also utilize the signal-to-noise-ratio metric to exclude a section from the base-calling process and/or to exclude nucleotide-base calls generated for that section from the nucleotide-base-call data 112. Further, the signal-to-noise-aware base calling system 106 can utilize the signal-to-noise-ratio metric determined for a section of a nucleotide-sample slide to generate a quality metric corresponding to a nucleotide-base call generated for the signal detected from that section. In some instances, the signal-to-noise-aware base calling system 106 contributes additional information—such as the signal-to-noise-ratio metrics themselves, the signal-to-noise-ratio threshold used for filtering, the average quality metric, etc.—to the nucleotide-base-call data 112.

As further indicated by FIG. 1, the server device(s) 102 may generate, receive, analyze, store, and transmit digital data, such as data related to nucleotide-base calls or sequencing nucleic-acid polymers. As shown in FIG. 1, the sequencing device 110 may send (and the server device(s) 102 may receive) nucleotide-base-call data 112 from the sequencing device 110. The server device(s) 102 may also communicate with the user client device 114. In particular, the server device(s) 102 can send nucleobase sequences, error data, and other information to the user client device 114.

In some embodiments, the server device(s) 102 comprise a distributed collection of servers where the server device(s) 102 include a number of server devices distributed across the network 108 and located in the same or different physical locations. Further, the server device(s) 102 can comprise a content server, an application server, a communication server, a web-hosting server, or another type of server.

As further shown in FIG. 1, the server device(s) 102 can include a sequencing system 104. Generally, the sequencing system 104 analyzes the nucleotide-base-call data 112 received from the sequencing device 110 to determine nucleotide-base sequences for nucleic-acid polymers, such as the nucleotide-base sequence for a sample genome. For example, the sequencing system 104 can receive raw data from the sequencing device 110 and determine a nucleotide-base sequence for a nucleic-acid segment. In some embodiments, the sequencing system 104 determines the sequences of nucleotide-bases in DNA and/or RNA segments or oligonucleotides. In some cases, as mentioned above, the sequencing system 104 receives pre-processed data that includes nucleotide-base calls, error/accuracy information in the form of quality metrics, and or data regarding filtered (e.g., excluded) clusters. Accordingly, in some implementations, the sequencing system 104 organizes data from the nucleotide-base-call data 112 into a useful, user-readable format.

As further illustrated in FIG. 1, the signal-to-noise-aware base calling system 106 may be located on the sequencing device 110 and/or on the server device(s) 102 as part of the sequencing system 104. Accordingly, in some embodiments, the signal-to-noise-aware base calling system 106 is implemented by (e.g., located entirely or in part) on the server device(s) 102. In yet other embodiments, the signal-to-noise-aware base calling system 106 is implemented by one or more other components of the environment 100, such as the sequencing device 110. In particular, the signal-to-noise-aware base calling system 106 can be implemented in a variety of different ways across the server device(s) 102, the network 108, and the sequencing device 110.

As further illustrated and indicated in FIG. 1, the user client device 114 can generate, store, receive, and send digital data. In particular, the user client device 114 can receive sequencing data from the server device(s) 102 or the sequencing device 110. Furthermore, the user client device 114 may communicate with the server device(s) 102 to receive nucleobase sequences as well as reports of irregularities within a sequencing cycle. The user client device 114 can accordingly present sequencing data and notifications of nucleobase calls within a graphical user interface to a user associated with the user client device 114. In some cases, the user client device 114 can further present intensity-value boundaries, nucleotide-base-call data, and other information related to computation and use of signal-to-noise-ratio metrics for display.

The user client device 114 illustrated in FIG. 1 may comprise various types of client devices. For example, in some embodiments, the user client device 114 includes non-mobile devices, such as desktop computers or servers, or other types of client devices. In yet other embodiments, the user client device 114 includes mobile devices, such as laptops, tablets, mobile telephones, or smartphones. Additional details with regard to the user client device 114 are discussed below with respect to FIG. 12.

As further illustrated in FIG. 1, the user client device 114 includes a sequencing application 116. The sequencing application 116 may be a web application or a native application stored and executed on the user client device 114 (e.g., a mobile application, desktop application). The sequencing application 116 can receive data from the signal-to-noise-aware base calling system 106 and can present, for display at the user client device 114, sequencing data. Furthermore, the sequencing application 116 can provide notifications regarding intensity-value boundaries, filtered nucleotide-base calls, etc. In some implementations, the signal-to-noise-aware base calling system 106 is located on the user client device 114 as part of the sequencing application 116.

Though FIG. 1 illustrates the components of environment 100 communicating via the network 108, in certain implementations, the components of environment 100 can also communicate directly with each other, bypassing the network 108. For instance, and as previously mentioned, in some implementations, the server device(s) 102 communicates directly with the sequencing device 110 and/or the user client device 114. Moreover, the signal-to-noise-aware base calling system 106 can access one or more databases housed on or accessed by the server device(s) 102 or elsewhere in the environment 100.

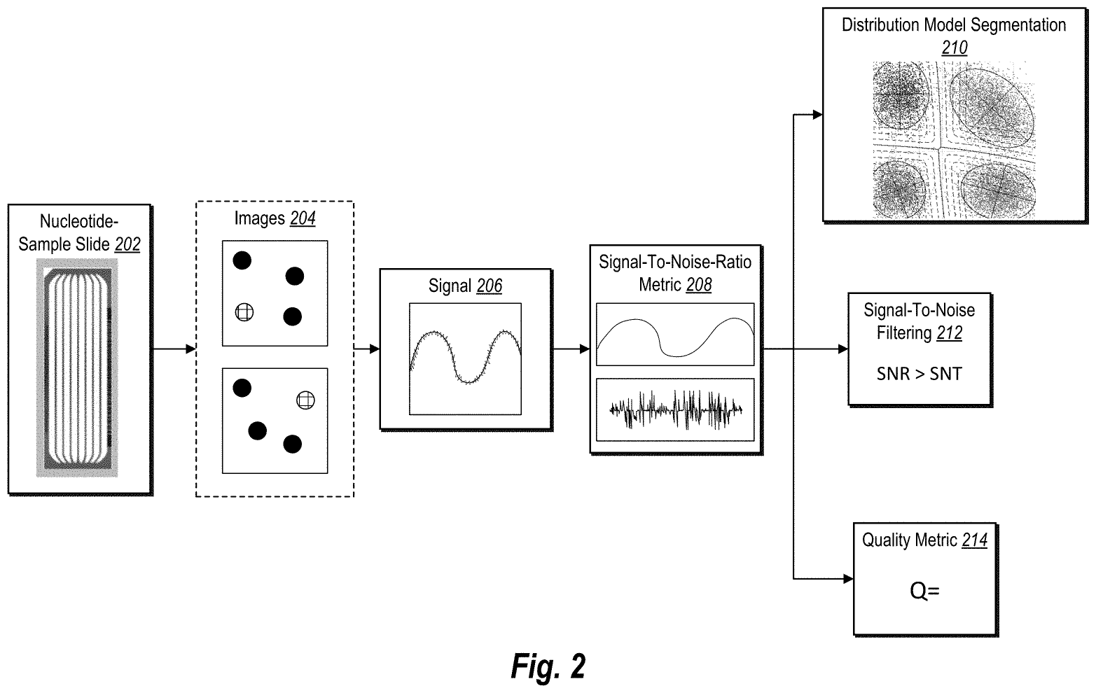

As previously mentioned, the signal-to-noise-aware base calling system 106 generates a signal-to-noise-ratio metric for a section of a nucleotide-sample slide. In particular, the signal-to-noise-aware base calling system 106 generates a signal-to-noise-ratio metric for a signal detected from labeled nucleotide bases located at or within the section. The signal-to-noise-aware base calling system 106 can utilize the signal-to-noise-ratio metric to provide various nucleotide-base-calling features. FIG. 2 illustrates an overview diagram of the signal-to-noise-aware base calling system 106 generating and utilizing a signal-to-noise-ratio metric in accordance with one or more embodiments.

As shown in FIG. 2, the signal-to-noise-aware base calling system 106 utilizes a nucleotide-sample slide 202 for sequencing. The nucleotide-sample slide 202 can include oligonucleotides that receive or incorporate labeled nucleotide bases. In particular, the nucleotide-sample slide 202 can include a cluster of oligonucleotides within each section (e.g., well). When stimulated, the labeled nucleotide bases can emit a signal having characteristics associated with the type of nucleotide base.

As further shown in FIG. 2, the signal-to-noise-aware base calling system 106 captures images 204 of at least one section of the nucleotide-sample slide 202. In particular, the signal-to-noise-aware base calling system 106 captures the images 204 as the labeled nucleotide bases within the section of the nucleotide-sample slide 202 emit a signal. As shown, in one or more embodiments, the signal-to-noise-aware base calling system 106 captures multiple images. For example, the signal-to-noise-aware base calling system 106 can capture multiple images using various image filters. To illustrate, in some embodiments, the signal-to-noise-aware base calling system 106 utilizes a two-channel implementation, capturing two images of the section of the nucleotide-sample slide 202. In particular, the signal-to-noise-aware base calling system 106 captures a first image using a first image using a first image filter and captures a second image using a second image filter. The first and second images can capture an intensity of the emitted signal that corresponds to the image filter used. In some cases, the signal-to-noise-aware base calling system 106 utilizes a four-channel implementation and captures four different images of the section of the nucleotide-sample slide 202. Similar to the two-channel implementation, the signal-to-noise-aware base calling system 106 can capture each image for the four-channel implementation using a different image filter. Each image can capture an intensity of the emitted signal based on the image filter used for that image. Thus, in some cases, each of the four images depicts the emitted signal with a different intensity.

As indicated in FIG. 2, the images 204 portray a signal 206 emitted from the labeled nucleotide bases located within the section of the nucleotide-sample slide 202. As previously mentioned, the signal 206 can indicate the type of nucleotide base that was added to oligonucleotides within the section of the nucleotide-sample slide 202. For example, as will be discussed in more detail below, the signal 206 can have one or more corresponding intensity values that indicate the type of nucleotide base. To illustrate, in some implementation, each of the images 204 captures at least one intensity value corresponding to the signal 206.

The signal 206 can have some associated noise. In particular, the signal 206 can have an associated noise level that affects the purity of the signal 206. Accordingly, as indicated by FIG. 2, the signal-to-noise-aware base calling system 106 can generate a signal-to-noise-ratio metric 208 for the signal 206. For example, the signal-to-noise-aware base calling system 106 can determine a scaling factor corresponding to the signal 206. In one or more embodiments, the signal-to-noise-aware base calling system 106 equates the determined scaling factor to the signal 206. Further, the signal-to-noise-aware base calling system 106 can determine a noise level corresponding to the signal 206. Accordingly, the signal-to-noise-aware base calling system 106 can generate the signal-to-noise-ratio metric 208 for the signal 206 utilizing the scaling factor and the noise level.

The signal-to-noise-aware base calling system 106 can utilize the signal-to-noise-ratio metric 208 for providing various base-calling features. For example, as shown in FIG. 2, the signal-to-noise-aware base calling system 106 can utilize the signal-to-noise-ratio metric 208 for distribution-model segmentation 210. In particular, the signal-to-noise-aware base calling system 106 can utilize the signal-to-noise-ratio metric 208 to segment a base-call-distribution model—such as a Gaussian mixture model—into separate base-call-distribution models. In some implementations, the signal-to-noise-aware base calling system 106 segments the base-call-distribution model by fitting a separate base-call-distribution model to each of a plurality of signal-to-noise-ratio ranges. Indeed, as will be discussed further below, the signal-to-noise-aware base calling system 106 can determine signal-to-noise-ratio metrics (including the signal-to-noise-ratio metric 208) for a plurality of signals detected from a plurality of sections of the nucleotide-sample slide 202. The signal-to-noise-aware base calling system 106 further determines a plurality of signal-to-noise-ratio ranges for the plurality of signal-to-noise-ratio metrics. Accordingly, the signal-to-noise-aware base calling system 106 can fit a base-call distribution to each of the signal-to-noise-ratio ranges.

The signal-to-noise-aware base calling system 106 can further utilize the base-call-distribution model for a particular signal-to-noise-ratio range to generate a nucleotide-base call for the signals having a signal-to-noise-ratio metric that falls within that range. Thus, the signal-to-noise-aware base calling system 106 can utilize the signal-to-noise-ratio metric 208 to generate a nucleotide-base call for the signal 206 via the distribution-model segmentation 210.

As further shown in FIG. 2, the signal-to-noise-aware base calling system 106 can utilize the signal-to-noise-ratio metric 208 for signal-to-noise filtering 212. In particular, the signal-to-noise-aware base calling system 106 can establish a signal-to-noise-ratio threshold and exclude the signal 206 (e.g., the corresponding section of the nucleotide-sample slide 202) from nucleotide-base-call data if the signal-to-noise-ratio metric 208 fails to satisfy the signal-to-noise-ratio threshold.

Additionally, as further shown in FIG. 2, the signal-to-noise-aware base calling system 106 can utilize the signal-to-noise-ratio metric 208 to determine a quality metric 214 for a nucleotide-base call generate for the signal 206. For example, the signal-to-noise-aware base calling system 106 can utilize a base-call-quality model to determine the quality metric 214 based on the signal-to-noise-ratio metric 208.

Though much of the above discussion (as well as the following discussion) focuses on determining a signal-to-noise-ratio metric for a section of a nucleotide-sample slide, it should be understood that the signal-to-noise-aware base calling system 106 can determine a signal-to-noise-ratio metric for each of a plurality of sections of the nucleotide-sample slide in parallel. For instance, in one or more embodiments, the signal-to-noise-aware base calling system 106 detects a signal from each section of the nucleotide-sample slide (e.g., each well or each section corresponding to a cluster) and determines a signal-to-noise-ratio metric for each detected signal. Thus, the signal-to-noise-aware base calling system 106 can utilize the various signal-to-noise-ratio metrics for determining nucleotide-base calls via segmented base-call-distribution models, signal-to-noise filtering, and determining quality metrics for generated nucleotide-base calls.

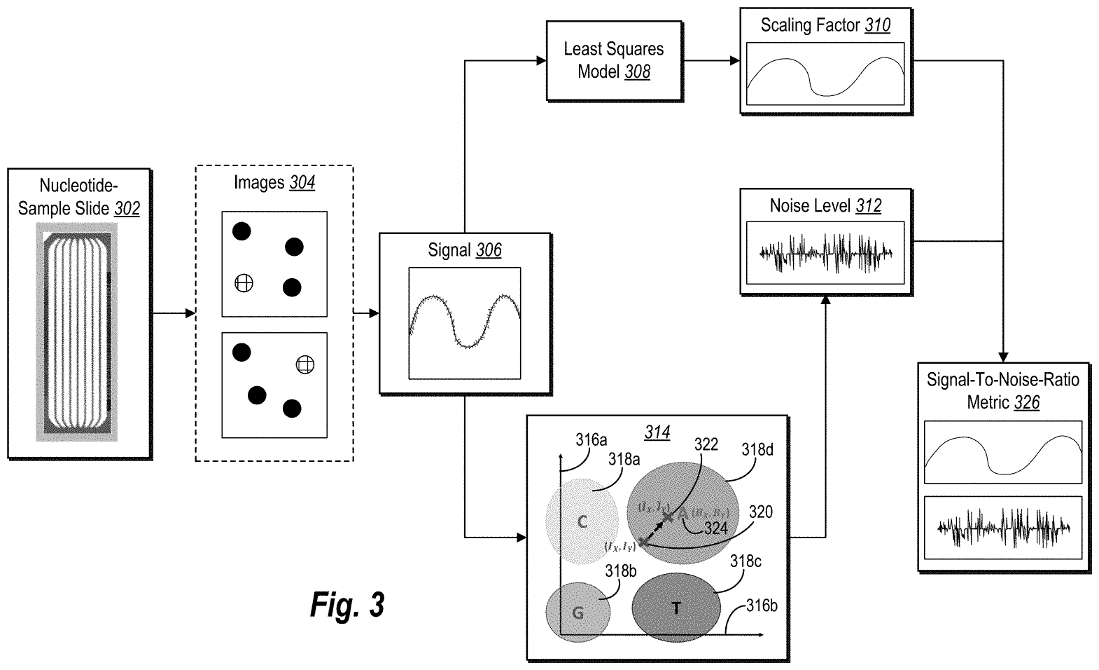

As previously mentioned, in one or more embodiments, the signal-to-noise-aware base calling system 106 determines a signal-to-noise-ratio metric for a signal detected from labeled nucleotide bases within a section of a nucleotide-sample slide. FIG. 3 illustrates a diagram for determining a signal-to-noise-ratio metric in accordance with one or more embodiments.

As shown in FIG. 3, the signal-to-noise-aware base calling system 106 captures images 304 of at least one section of a nucleotide-sample slide 302. For instance, a camera for the sequencing device 110—and associated with the signal-to-noise-aware base calling system 106—captures the images 304 of tiles within the nucleotide-sample slide 302, where each tile includes multiple nanowells comprising clusters or multiple subsections comprising clusters. As further shown, the images 304 portray a signal 306 emitted from the at least one section of the nucleotide-sample slide 302 (e.g., from the labeled nucleotide bases within a well or subsection corresponding to a cluster.)

As further shown in FIG. 3, the signal-to-noise-aware base calling system 106 determines a scaling factor 310 corresponding to the signal 306. In particular, the signal-to-noise-aware base calling system 106 determines the scaling factor 310 utilizing a least squares model 308. In one or more embodiments, the signal-to-noise-aware base calling system 106 utilizes the least squares model 308 to determine variation correction coefficients corresponding to the signal 306. In one or more embodiments, such as where a two-channel implementation is used, the variation correction coefficients include the scaling factor 310 that accounts for scale variation in an inter-cluster intensity profile and two offset factors (also referred to as channel-specific offset coefficients) that account for shift variation along the first and second intensity channels in the inter-cluster intensity profile variation, respectively.

The signal-to-noise-aware base calling system 106 can utilize the least squares model 308 to determine the variation correction coefficients by determining a relationship between a measured intensity for the labeled nucleotide bases (e.g., a measured intensity corresponding to the signal 306) and the variation correction coefficients. The signal-to-noise-aware base calling system 106 can further determine an error function based on the relationship between the measured intensity and the variation correction coefficients. The signal-to-noise-aware base calling system 106 can determine the scaling factor 310 by generating a partial derivative of the error function with respect to the scaling factor. In particular, in some implementations, the 106 utilizes the least squares model 308 to determine two partial derivatives of the error function: one with respect to the scaling factor 310 and another with respect to the channel-specific offset factors. Indeed, in some implementations, the signal-to-noise-aware base calling system 106 utilizes the least squares model 308 to determine the scaling factor 310 as described in U.S. Patent Application No. 63/106,256, filed Oct. 27, 2020, and entitled “SYSTEMS AND METHODS FOR PRE-CLUSTER INTENSITY CORRECTION AND BASE CALLING,” which is incorporated herein by reference in its entirety.

As further shown in FIG. 3, the signal-to-noise-aware base calling system 106 determines a noise level 312 corresponding to the signal 306. In particular, as shown, the signal-to-noise-aware base calling system 106 can determine the noise level 312 using corrected intensity values for the section of the nucleotide-sample slide 302 (e.g., for the signal 306). In one or more embodiments, the term “corrected intensity value” refers to an intensity value corresponding to a signal emitted from a section of a nucleotide-sample slide that has been adjusted based on one or more features of the signal. To illustrate, in one or more embodiments, a corrected intensity value includes an intensity value that has been corrected to account for offset and a scaling factor corresponding to an intensity value. Upon correction, in some cases, the corrected intensity value is closer to a centroid of a nucleotide cloud than the corresponding intensity value that was initially measured for the signal. For example, in a two-channel implementation, the signal-to-noise-aware base calling system 106 can determine a pair of corrected intensity values (e.g., one for each intensity channel) so that the pair is nearer to the centroid of a nucleotide cloud than the corresponding pair of intensity values initially measured for the signal. In one or more embodiments, the signal-to-noise-aware base calling system 106 determines the corrected intensity values using the following:

$${\check{I}}_{X} = \frac{( {I}_{X} - {O}_{X} )}{S} {\check{I}}_{Y} = \frac{( {I}_{Y} - {O}_{Y} )}{S} \qquad ( 1 )$$

In function (1), I_{X} and I_{Y} represent the corrected intensity values, and I_{X} and I_{Y} represent the intensity values initially measured for the signal 306. Further, S represents a scaling factor determined for the signal 306 (e.g., the scaling factor 310) and O_{X} and O_{Y} represent the offset factors corresponding to the signal 306. In a four-channel implementation, the signal-to-noise-aware base calling system 106 similarly operates to determine four corrected intensity values (e.g., one for each of the four intensity channels used). In such a case, the signal-to-noise-aware base calling system 106 utilizes a function similar to function (1) to determine the corrected intensity values by incorporating their respective offset factors. In particular, the signal-to-noise-aware base calling system 106 can determine a corrected intensity value for a given intensity channel using the intensity value initially measured for that intensity channel, the offset factor determined for that intensity channel, and the scaling factor.

FIG. 3 provides a visualization of the corrected intensity values via graph 314. The axes 316a-316b of the graph 314 represent intensity values for each intensity channel in a two-channel implementation. The graph 314 maps nucleotide clouds 318a-318d to the intensity values with their respective intensity-value boundaries. As shown in FIG. 3, the intensity values initially measured for the signal 306 correspond to the point 320 within the nucleotide cloud 318d. Further, the corrected intensity values correspond to the point 322. As further shown, the point 322 corresponding to the corrected intensity values is nearer to the centroid 324 of the nucleotide cloud 318d.

In one or more embodiments, the signal-to-noise-aware base calling system 106 determines the noise level 312 by determining the distance between the corrected intensity values and centroid intensity values of a nucleotide cloud, such as the nearest nucleotide cloud or the nearest centroid. For example, in one or more embodiments, the signal-to-noise-aware base calling system 106 determines the noise level 312 as follows, where B_{X} and B_{Y} represent the centroid intensity values:

Noise={square root over (((I_{X}−B_{X})²+(I_{Y}−B_{Y})²))} (2)

In one or more embodiments, the signal-to-noise-aware base calling system 106 further determines the noise level 312 using the noise level determined for the same section of the nucleotide-sample slide 302 determined for one or more previous sequencing cycles. Indeed, in some implementations, the signal-to-noise-aware base calling system 106 stores the noise levels determined for the section of the nucleotide-sample slide 302 after each sequencing cycle. In one or more embodiments, the signal-to-noise-aware base calling system 106 averages the stored noise levels for the previous sequencing cycles and utilizes the averaged noise level in determining the noise level 312 for the current sequencing cycle (e.g., by adding the averaged noise level to the noise level determined using function 2, by averaging the averaged noise level with the noise level determined using function 2, etc.). In some implementations, the signal-to-noise-aware base calling system 106 utilizes a weighted average of the noise levels for the previous sequencing cycles. For example, the signal-to-noise-aware base calling system 106 can assign weights to the noise levels determined for the previous sequencing cycles based on recency. To illustrate, the signal-to-noise-aware base calling system 106 can assign relatively higher weights to the noise levels determined for more recent sequencing cycles.

In some implementations, the signal-to-noise-aware base calling system 106 utilizes noise levels for a set number of previous sequencing cycles in determining the noise level for the current sequencing cycle. For example, the signal-to-noise-aware base calling system 106 can determine the set number of previous sequencing cycles to utilize based on user input. In some cases, the signal-to-noise-aware base calling system 106 utilizes the noise levels for all previous sequencing cycles (e.g., all noise levels within the same read or across multiple reads).

Though the paragraphs above describe using previous noise levels associated with a section of a nucleotide-sample slide for determining the noise level for that section for a current sequencing cycle, in some cases, the signal-to-noise-aware base calling system 106 utilizes the previous noise levels associated with all sections of the nucleotide-sample slide.

As shown in FIG. 3, the signal-to-noise-aware base calling system 106 utilizes the scaling factor 310 and the noise level 312 to determine a signal-to-noise-ratio metric 326 for the signal 306. For example, the signal-to-noise-aware base calling system 106 can determine the signal-to-noise-ratio metric 326 utilizing a ratio of the scaling factor 310 to the noise level 312. Indeed, in one or more embodiments, the signal-to-noise-aware base calling system 106 equates scaling factor 310 to the signal 306 (e.g., treats the scaling factor 310 as the signal 306) for purposes of determining the signal-to-noise-ratio metric 326.

In one or more embodiments, the signal-to-noise-aware base calling system 106 accounts for phasing or pre-phasing when determining the signal-to-noise-ratio metric for a signal. As used herein, the term “phasing” refers to an effect or situation where sequencing on one molecule falls at least one base behind other molecules at a particular cycle. Conversely, as used herein, the term “pre-phasing” refers to an effect or situation where sequencing on one molecule jumps at least one base ahead of other molecules at a particular cycle. In one or more embodiments, to correct for the effects of phasing or pre-phasing, the signal-to-noise-aware base calling system 106 can detect a signal with an intensity value for base incorporation at each cycle and correct the intensity value by (i) subtracting an intensity value of an immediately previous cycle from an intensity value of a current cycle and (ii) subtracting an intensity value of an immediately subsequent cycle from the intensity value of the current cycle. Indeed, in one or more embodiments, the signal-to-noise-aware base calling system 106 corrects the effects of phasing or pre-phasing as described in U.S. Pat. No. 10,689,696, issued Jun. 23, 2020, and entitled “Methods and Systems for Analyzing Image Data,” which is incorporated herein by reference in its entirety.

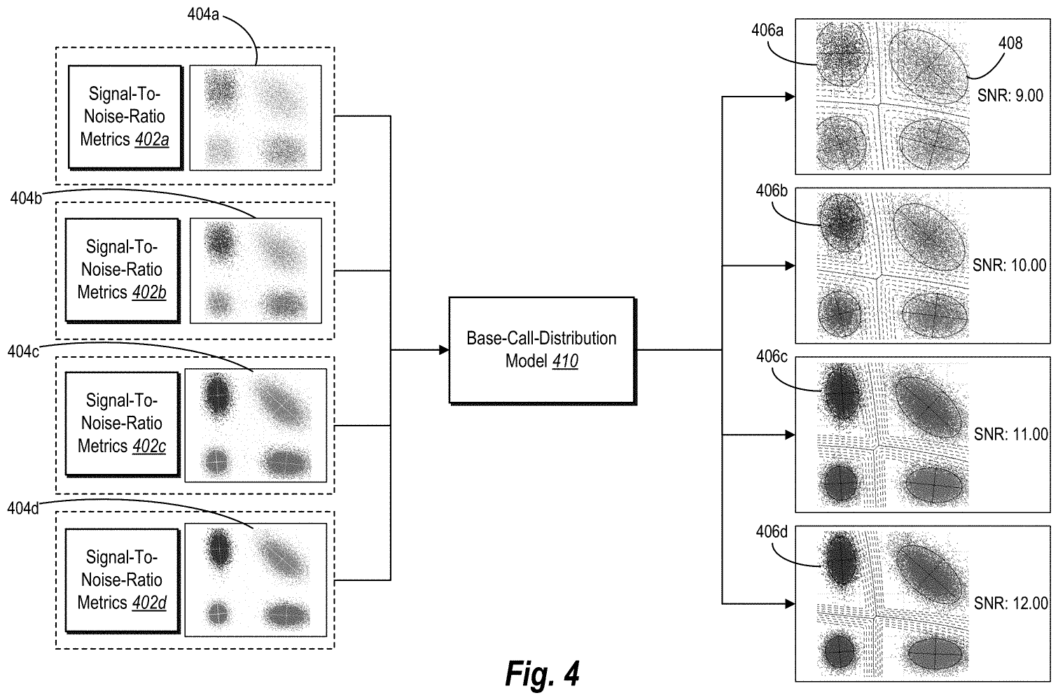

As previously discussed, in one or more embodiments, the signal-to-noise-aware base calling system 106 utilizes the signal-to-noise-ratio metrics corresponding to signals detected from a plurality of sections of a nucleotide-sample slide for distribution-model segmentation. FIG. 4 illustrates a block diagram of utilizing signal-to-noise-ratio metrics for distribution-model segmentation in accordance with one or more embodiments.

As shown in FIG. 4, the signal-to-noise-aware base calling system 106 determines the signal-to-noise-ratio metrics 402a-402d. In particular, the signal-to-noise-aware base calling system 106 determines a signal-to-noise-ratio metric for a plurality of sections of a nucleotide-sample slide based on the signals detected from those sections during a sequencing cycle. The signal-to-noise-aware base calling system 106 can determine the signal-to-noise-ratio metrics as discussed above with reference to FIG. 3.

As further shown in FIG. 4, the signal-to-noise-aware base calling system 106 separates the signal-to-noise-ratio metrics 402a-402d into different groups. For example, the signal-to-noise-aware base calling system 106 can separate the signal-to-noise-ratio metrics 402a-402d utilizing signal-to-noise-ratio ranges. Indeed, in one or more embodiments, the signal-to-noise-aware base calling system 106 establishes multiple signal-to-noise-ratio ranges. The signal-to-noise-aware base calling system 106 can establish the signal-to-noise-ratio ranges based on user input, using fixed ranges, or based on the signal-to-noise-ratio metrics determined for the current sequencing cycle (e.g., establish a first range that covers the lowest set of signal-to-noise-ratio metrics, establish a second range that covers the second-lowest set of signal-to-noise-ratio metrics, etc.). Though FIG. 4 illustrates a particular number of signal-to-noise-ratio ranges, the signal-to-noise-aware base calling system 106 can establish various numbers of signal-to-noise-ratio ranges.

In one or more embodiments, each of the signal-to-noise-ratio metrics 402a-402d correspond to a different signal-to-noise-ratio range. For example, the signal-to-noise-ratio metrics 402a can correspond to a first signal-to-noise-ratio range (e.g., 9.00-9.99), the signal-to-noise-ratio metrics 402b can correspond to a second signal-to-noise-ratio range (e.g., 10.00-10.99), the signal-to-noise-ratio metrics 402c can correspond to a third signal-to-noise-ratio range (e.g., 11.00-11.99), and the signal-to-noise-ratio metrics 402d can correspond to a fourth signal-to-noise-ratio range (e.g., 12.00-12.99). The signal-to-noise-aware base calling system 106 can associate the signal detected from each section of the nucleotide-sample slide with the signal-to-noise-ratio range within which the signal's corresponding signal-to-noise-ratio metric falls. Indeed, as shown in FIG. 4, the signal-to-noise-aware base calling system 106 establishes the sets of intensity values 404a-404d based on the signal-to-noise-ratio ranges. For example, the set of intensity values 404a includes intensity values for the signals associated with the signal-to-noise-ratio metrics 402a (e.g., associated with the first signal-to-noise-ratio range that includes the signal-to-noise-ratio metrics 402a).

As further shown, the signal-to-noise-aware base calling system 106 generates intensity-value boundaries for the signals from the sections of the nucleotide-sample slide. For example, FIG. 4 illustrates graphs 406a-406d having sets of intensity-value boundaries (e.g., intensity-value boundary 408) corresponding to each possible nucleotide base (e.g., A, T, C, or G).

In one or more embodiments, the signal-to-noise-aware base calling system 106 generates the sets of intensity-value boundaries in accordance with one or more base-call-distribution models. For example, the signal-to-noise-aware base calling system 106 can generate a first set of intensity-value boundaries (e.g., those shown in the graph 406a) in accordance with a first base-call-distribution model, a second set of intensity-value boundaries (e.g., those shown in the graph 406b) in accordance with a second base-call-distribution model, etc.

As shown in FIG. 4, the signal-to-noise-aware base calling system 106 can utilize a base-call-distribution model 410 to generate the intensity-value boundaries. In some cases, the base-call-distribution model 410 includes a single base-call-distribution model, but the signal-to-noise-aware base calling system 106 can utilize multiple base-call-distribution models in some implementations (e.g., a separate base-call-distribution model for each signal-to-noise-ratio range). Further, the base-call-distribution model 410 can include a Gaussian distribution model in one or more embodiments, though other base-call-distribution models can be utilized as well.

Though not shown in FIG. 4, the signal-to-noise-aware base calling system 106 can generate a nucleotide-base call for a signal utilizing one of the sets of intensity-value boundaries. In particular, the signal-to-noise-aware base calling system 106 can generate the nucleotide-base call utilizing the set of intensity-value boundaries corresponding to the signal-to-noise-ratio range associated with the signal (i.e., in accordance with the base-call-distribution model corresponding to the signal-to-noise-ratio range). In one or more embodiments, the signal-to-noise-aware base calling system 106 further generates the nucleotide-base call utilizing the intensity values determined for the signal.

To illustrate, upon determining that a signal had a corresponding signal-to-noise-ratio metric that fell within a first signal-to-noise-ratio range (e.g., 9.00-9.99), the signal-to-noise-aware base calling system 106 can use the set of intensity-value boundaries generated for the first signal-to-noise-ratio range (e.g., those shown in the graph 406a) to generate the nucleotide-base call. The signal-to-noise-aware base calling system 106 can further determine how the set of intensity values for the signal relate to the set of intensity-value boundaries and generate the nucleotide-base call accordingly. For example, upon determining that the set of intensity values for the signal fall within the decision boundaries for a particular nucleotide base, the signal-to-noise-aware base calling system 106 can generate a nucleotide-base call indicating that the signal is associated with that nucleotide base. Based on determining that the set of intensity values for the signal fall outside the decision boundaries for all nucleotide bases, the signal-to-noise-aware base calling system 106 can generate the nucleotide-base call for the signal based on a proximity the decision boundary for each nucleotide base and/or based on a proximity to the centroid of the nucleotide cloud corresponding to each nucleotide base.

Because the signal-to-noise-aware base calling system 106 generates a nucleotide-base call for a signal in accordance with the base-call-distribution model that corresponds to the signal-to-noise-ratio range associated with the signal, the signal-to-noise-aware base calling system 106 can generate different nucleotide-base calls for signals having similar intensity values in some cases. To illustrate, in one or more embodiments, the signal-to-noise-aware base calling system 106 generates, for a first signal-to-noise-ratio range, a first set of intensity-value boundaries corresponding to the different nucleotide bases according to a first base-call-distribution model. The signal-to-noise-aware base calling system 106 further generates, for a second signal-to-noise-ratio range, a second set of intensity-value boundaries corresponding to the different nucleotide bases according to a second base-call-distribution model, the second set of intensity-value boundaries differing from the first set of intensity-value boundaries.

Further, the signal-to-noise-aware base calling system 106 can detect a first signal corresponding to a first signal-to-noise-ratio metric within the first signal-to-noise-ratio range and having a set of intensity values outside of the first set of intensity-value boundaries and outside the second set of intensity-value boundaries and detect a second signal corresponding to a second signal-to-noise-ratio metric within the second signal-to-noise-ratio range and having the set of intensity values (e.g., the same set of intensity values as the first signal). Accordingly, the signal-to-noise-aware base calling system 106 can generate a first nucleotide-base call for the first signal based on the first set of intensity-value boundaries for the first base-call-distribution model and generate a second nucleotide-base call for the second signal based on the second set of intensity-value boundaries for the second base-call-distribution model. Indeed, even though the two signals have the same set of intensity values, the signal-to-noise-aware base calling system 106 can generate different nucleotide-base calls utilizing the two different base-call-distribution models.

By generating intensity-value boundaries for various signal-to-noise-ratio ranges, the signal-to-noise-aware base calling system 106 operates more flexibly when compared to conventional sequencing platforms. Indeed, the signal-to-noise-aware base calling system 106 tailors the intensity-value boundaries to characteristics—such as the signal-to-noise-ratio metrics—of detected signals, providing more flexibility than conventional platforms, which tend to utilize the same set of decision boundaries for all signals regardless of their characteristics. By tailoring the intensity-value boundaries as described, the signal-to-noise-aware base calling system 106 further operates more accurately than the conventional sequencing platforms. In particular, the signal-to-noise-aware base calling system 106 generates nucleotide-base calls for signals using intensity-value boundaries that are more appropriate for those signals as the intensity-value boundaries correspond more closely to the characteristics of the signals.

Further, by generating different intensity-value boundaries for different signal-to-noise-ratio ranges, the signal-to-noise-aware base calling system 106 more accurately determines the quality of a nucleotide-base call generated for detected signals. Indeed, as can be seen in FIG. 4, the graphs 406a-406d each include a set of dashed contour lines. The contour lines can represent different quality metrics (e.g., Q scores) that correspond to nucleotide-base calls. For example, the contour line located closest to a given intensity-boundary value can correspond to a quality metric that indicates a relatively high degree of confidence in the accuracy of a nucleotide-base call associated with the intensity-value boundary (e.g., a low probability of error) while contour lines further away correspond to quality metrics indicating relatively lower degrees of confidence. Thus, the contour lines associated with an intensity-value boundary indicate that intensity values farther way from an intensity-value boundary correspond to lower degrees of confidence if assigned a nucleotide-base call corresponding to the intensity-value boundary.

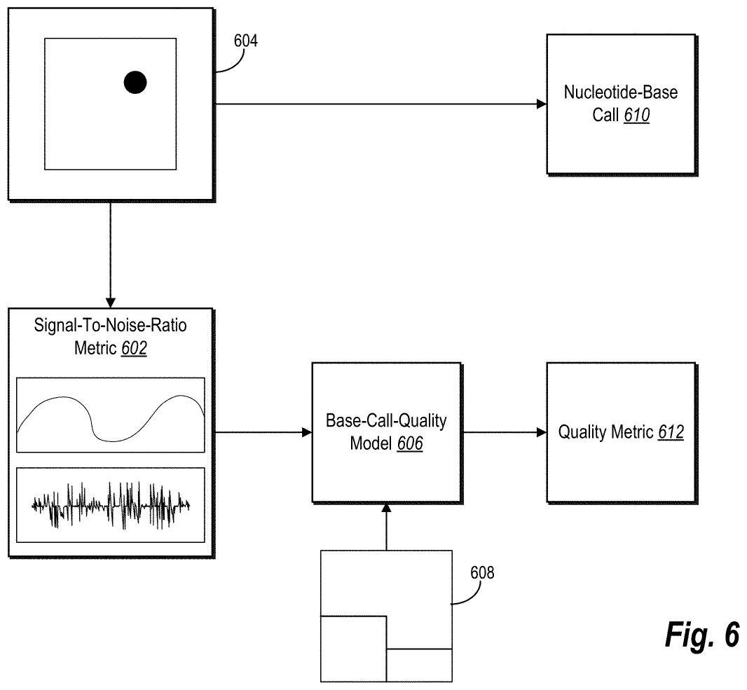

As can further be seen in FIG. 4, the set of dashed contour lines associated with the intensity-value boundaries change among the graphs 406a-406d (e.g., as the signal-to-noise-ratio range of a graph includes higher signal-to-noise-ratio metrics, the contour lines are closer together). Accordingly, as with the generation of a nucleotide-base call itself, the graphs 406a-406d indicate that the determination of the quality of a nucleotide-base call is also tailored to the characteristics of the corresponding signal. Thus, generating nucleotide-base calls using the separate intensity-value boundaries can lead to more accurate determinations of the quality of those nucleotide-base calls, which will be discussed further in more detail below with reference to FIG. 6.

FIG. 4 depicts generation of intensity-value boundaries and corresponding nucleotide-base calls in a two-channel implementation where two intensity channels are used. It should be noted, however, that the signal-to-noise-aware base calling system 106 can similarly operate in a four-channel implementation where four intensity channels are used. For example, in some implementations, the base-call-distribution model utilized to generate intensity-value boundaries is configured to generate intensity-value boundaries in accordance with four intensity channels.

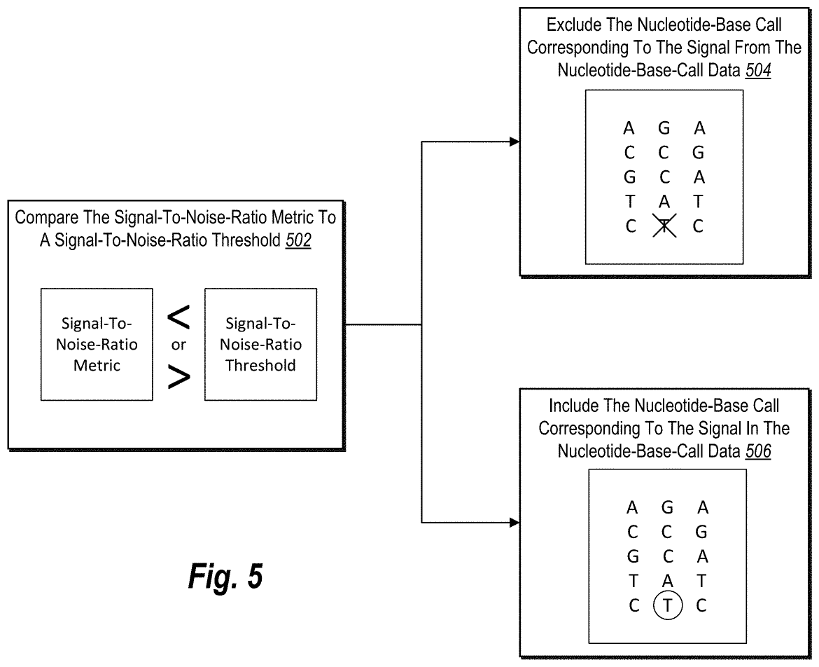

As further discussed above, in one or more embodiments, the signal-to-noise-aware base calling system 106 utilizes the signal-to-noise-ratio metric associated with a section of a nucleotide-sample slide to filter out one or more nucleotide-base calls generated for that section from the nucleotide-base-call data. FIG. 5 illustrates a block diagram of the signal-to-noise-aware base calling system 106 utilizing a signal-to-noise-ratio metric of a signal to filter nucleotide-base calls in accordance with one or more embodiments.

As shown in FIG. 5, the signal-to-noise-aware base calling system 106 performs an act of 502 of comparing the signal-to-noise-ratio metric determined for a signal to a signal-to-noise-ratio threshold. Indeed, in one or more embodiments, the signal-to-noise-aware base calling system 106 establishes a signal-to-noise-ratio threshold to be used filtering nucleotide-base calls. The signal-to-noise-aware base calling system 106 can establish the signal-to-noise-ratio threshold based on a user input or utilize a pre-determined signal-to-noise-ratio threshold. In some implementations, the signal-to-noise-aware base calling system 106 establishes the signal-to-noise-ratio threshold based on historical data. For example, the signal-to-noise-aware base calling system 106 can analyze previous sequencing data to determine which signal-to-noise-ratio metrics are typically associated with nucleotide-base calls that fall below a desired quality metric. Accordingly, the signal-to-noise-aware base calling system 106 can establish the signal-to-noise-ratio threshold high enough to filter out signals having such undesirable signal-to-noise-ratio metrics. In some cases, the signal-to-noise-aware base calling system 106 adjusts the signal-to-noise-ratio threshold with each sequencing cycle or series of sequencing cycles. In some instances, however, the signal-to-noise-aware base calling system 106 utilizes a constant signal-to-noise-ratio threshold through all sequencing cycles.

As further shown in FIG. 5, upon determining that the signal-to-noise-ratio metric fails to satisfy (e.g., is less than) the signal-to-noise-ratio threshold, the signal-to-noise-aware base calling system 106 performs an act 504 of excluding the nucleotide-base call corresponding to the signal from the nucleotide-base-call data. In particular, in some implementations, upon determining that the signal-to-noise-ratio metric corresponding to a signal fails to satisfy the signal-to-noise-ratio threshold, the signal-to-noise-aware base calling system 106 determines that the signal is of poor quality and the corresponding nucleotide-base call (if generated) is unreliable. Accordingly, the signal-to-noise-aware base calling system 106 excludes the nucleotide-base call from the nucleotide-base-call data.

In some implementations, the signal-to-noise-aware base calling system 106 further excludes, from the nucleotide-base-call data, one or more subsequent nucleotide-base calls generated for one or more subsequent signals detected from the same section of the nucleotide-sample slide. In other words, the signal-to-noise-aware base calling system 106 can exclude all nucleotide-base calls that are generated for that section of the nucleotide-sample slide during subsequent sequencing cycles. As noted above, the signal-to-noise-aware base calling system 106 can accordingly exclude all nucleotide-base calls—or does not continue determining nucleotide-base calls for—a cluster of oligonucleotides corresponding to a well of a patterned nucleotide-sample slide or a subsection of a non-patterned nucleotide-sample slide for the cluster. In some implementations, the signal-to-noise-aware base calling system 106 also excludes, from the nucleotide-base-call data, one or more previous nucleotide-base calls generated for that section of the nucleotide-sample slide.

Indeed, in one or more embodiments, upon determining that the signal-to-noise-ratio metric determined for a signal fails to satisfy the signal-to-noise-ratio threshold, the signal-to-noise-aware base calling system 106 filters out the corresponding section of the nucleotide-sample slide altogether. In other words, the signal-to-noise-aware base calling system 106 determines, based on the failure to satisfy the signal-to-noise-ratio threshold, that the corresponding section of the nucleotide-sample slide is of poor quality and unreliable. Accordingly, upon determining the failure to satisfy the signal-to-noise-ratio threshold, the signal-to-noise-aware base calling system 106 can remove the section of the nucleotide-sample slide from subsequent sequencing cycles (e.g., the signal-to-noise-aware base calling system 106 will not analyze the section in future cycles).

As shown in FIG. 5, upon determining that the signal-to-noise-ratio metric does satisfy (e.g., is equal to or greater than) the signal-to-noise-ratio threshold, the signal-to-noise-aware base calling system 106 performs an act 506 of including the nucleotide-base call corresponding to the signal in the nucleotide-base-call data. For example, the signal-to-noise-aware base calling system 106 can generate a nucleotide-base call for the signal and add the nucleotide-base call to the nucleotide-base-call data.

In one or more embodiments, the signal-to-noise-aware base calling system 106 compares the signal-to-noise-ratio metric determined for the section of the nucleotide-sample slide to the signal-to-noise-ratio threshold at every sequencing cycle. Thus, the signal-to-noise-aware base calling system 106 can determine, at any sequencing cycle, to exclude nucleotide-base calls generated for that section of the nucleotide-sample slide from the nucleotide-base-call data.

By filtering out certain nucleotide-base calls (or their corresponding sections of the nucleotide-sample slide entirely) using the signal-to-noise-ratio metric, the signal-to-noise-aware base calling system 106 operates more accurately than conventional sequencing platforms. Indeed, the signal-to-noise-aware base calling system 106 can more accurately identify poor-quality nucleotide-base calls (or poor-quality sections of the nucleotide-sample slide) when compared to conventional platforms, which often rely exclusively on chastity-based filtering. Indeed, as mentioned above, filtering based on chastity values can fail to identify problems that may be dormant in early sequencing cycles but manifest as sequencing progresses. Accordingly, conventional platforms that rely exclusively on chastity values for filtering tend to include erroneous nucleotide-base calls within the resulting nucleotide-base-call data. By utilizing the signal-to-noise-ratio metric for filtering, however, the signal-to-noise-aware base calling system 106 can more accurately identify poor-quality nucleotide-base calls and exclude them from the nucleotide-base-call data, providing more accurate sequencing results.

As mentioned above, in one or more embodiments, the signal-to-noise-aware base calling system 106 determines a quality metric estimating an error of a nucleotide-base call generated for a signal utilizing the signal-to-noise-ratio metric. FIG. 6 illustrates a block diagram for generating a quality metric for a nucleotide-base call in accordance with one or more embodiments.

As shown in FIG. 6, the signal-to-noise-aware base calling system 106 determines a signal-to-noise-ratio metric 602 corresponding to a signal captured with an image 604 (or multiple images). As further shown, the signal-to-noise-aware base calling system 106 generates a nucleotide-base call 610 for the signal. For example, the signal-to-noise-aware base calling system 106 can generate the nucleotide-base call 610 utilizing the signal-to-noise-ratio metric 602 in accordance with a base-call-distribution model as discussed above with reference to FIG. 3.

As further shown in FIG. 6, the signal-to-noise-aware base calling system 106 generates a quality metric 612 for the nucleotide-base call 610 to estimate an error of the nucleotide-base call 610. In particular, the signal-to-noise-aware base calling system 106 generates the quality metric 612 utilizing a base-call-quality model 606. In one or more embodiments, the base-call-quality model 606 accepts one or more dimensions (e.g., inputs) related to features of a signal and/or features of the corresponding section of a nucleotide-sample slide and generates a quality metric based on those dimensions. Accordingly, the signal-to-noise-aware base calling system 106 can provide the signal-to-noise-ratio metric 602 as one of the inputs to the base-call-quality model 606.

As shown in FIG. 6, and as previously mentioned, the base-call-quality model 606 can include a Phred algorithm (as indicated by the graph 608). Thus, the signal-to-noise-aware base calling system 106 can utilize the signal-to-noise-ratio metric 602 as one of the inputs to the Phred algorithm. Further, the signal-to-noise-aware base calling system 106 can utilize the Phred algorithm to generate a Q-score (i.e., a Phred quality score) that estimates the accuracy of the nucleotide-base call 610. In other words, the quality metric 612 can include the Q-score generated by the Phred algorithm.

In some cases, the signal-to-noise-aware base calling system 106 utilizes the quality metric determined for the nucleotide-base call corresponding to a signal to map the nucleotide-base call to a reference genome. In particular, the signal-to-noise-aware base calling system 106 can map the oligonucleotide located at the section of the nucleotide-sample slide emitting the signal to a reference genome. Accordingly, in one or more embodiments, the signal-to-noise-aware base calling system 106 detects a signal by detecting the signal from labeled nucleotide bases incorporated into a growing oligonucleotide at a genomic position later determined in alignment with a reference genome. Additionally, the signal-to-noise-aware base calling system 106 generates the signal-to-noise-ratio metric for the nucleotide-base call at the genomic position corresponding to the signal. Further, the signal-to-noise-aware base calling system 106 can determine the quality metric for the nucleotide-base call and utilize the quality metric to map the nucleotide-base call to the reference genome.

As indicated above, in some implementations, the signal-to-noise-aware base calling system 106 utilizes values in addition to the signal-to-noise-ratio metric for determining the quality metric for a nucleotide-base call. For example, in some cases, the signal-to-noise-aware base calling system 106 utilizes a chastity value corresponding to a signal in addition to the signal-to-noise-ratio metric. To illustrate, in some cases, the signal-to-noise-aware base calling system 106 determines a chastity value for a signal (e.g., for the corresponding section of the nucleotide-sample slide) based on distances between the intensity values for the signal and intensity values of a nearest centroid and between the intensity values for the signal and intensity values for at least one additional centroid. In some instances, the signal-to-noise-aware base calling system 106 utilizes the second-nearest centroid as the additional centroid. Accordingly, the signal-to-noise-aware base calling system 106 can generate, utilizing the base-call-quality model, the quality metric based on the signal-to-noise-ratio metric and the chastity value.

By utilizing a signal-to-noise-ratio metric corresponding to a signal to generate a quality metric for a nucleotide-base call corresponding to the signal, the signal-to-noise-aware base calling system 106 can estimate the quality of nucleotide-base calls more accurately when compared to conventional sequencing platforms. Indeed, by incorporating the signal-to-noise-ratio metric into the analysis, the signal-to-noise-aware base calling system 106 utilizes an additional indicator of quality. Accordingly, the signal-to-noise-aware base calling system 106 makes the determination of quality utilizing more information than conventional sequencing platforms.

As mentioned above, the signal-to-noise-aware base calling system 106 provides for improved filtering of poor-quality sections of a nucleotide-sample slide. In particular, the signal-to-noise-aware base calling system 106 more accurately identifies poor-quality sections and excludes corresponding nucleotide-base calls from being generated or included in the nucleotide-base-call data. Thus, the signal-to-noise-aware base calling system 106 provides more accurate sequencing results when compared to conventional sequencing platforms, which may fail to identify problematic sections of the nucleotide-sample slide.

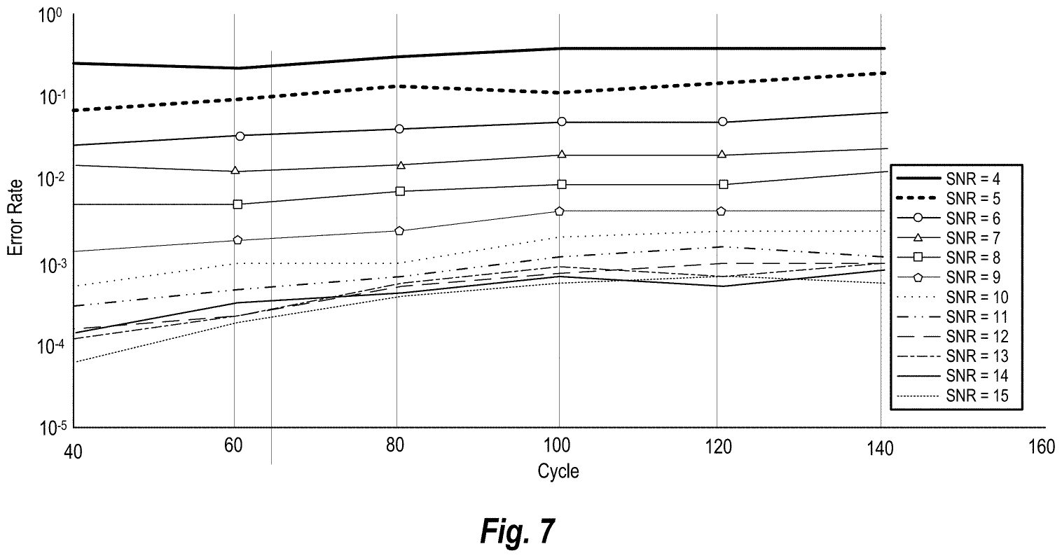

Researchers conducted studies to determine nucleotide-base-call error rates of sections of a nucleotide-sample slide associated with various signal-to-noise-ratio metrics. In particular, the researchers analyzed the nucleotide-base-call error rates across a series of sequencing cycles. FIG. 7 illustrates a graph showing the nucleotide-base-call error rates of sections of one or more nucleotide-sample slides having various signal-to-noise-ratio metrics in accordance with one or more embodiments.

As shown by the graph of FIG. 7, the tested sections of the one or more nucleotide-sample slides associated with lower signal-to-noise-ratio metrics (e.g., SNR=4, SNR=5, etc.) exhibit high error rates for the nucleotide-base calls. Comparatively, sections associated with higher signal-to-noise-ratio metrics (e.g., SNR=15, SNR=14, etc.) are associated with relatively lower error rates for the nucleotide-base calls. Thus, by excluding, from nucleotide-base-call data, the nucleotide-base calls associated with sections with lower signal-to-noise-ratio metrics, the signal-to-noise-aware base calling system 106 prevents inclusion of high-error data within the nucleotide-base-call data. Thus, the signal-to-noise-aware base calling system 106 provides more accurate, reliable base calls in the nucleotide-base-call data.

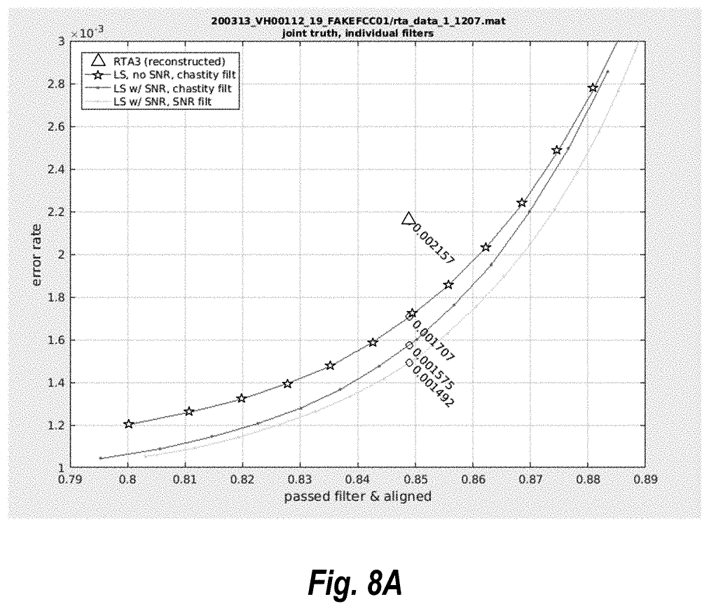

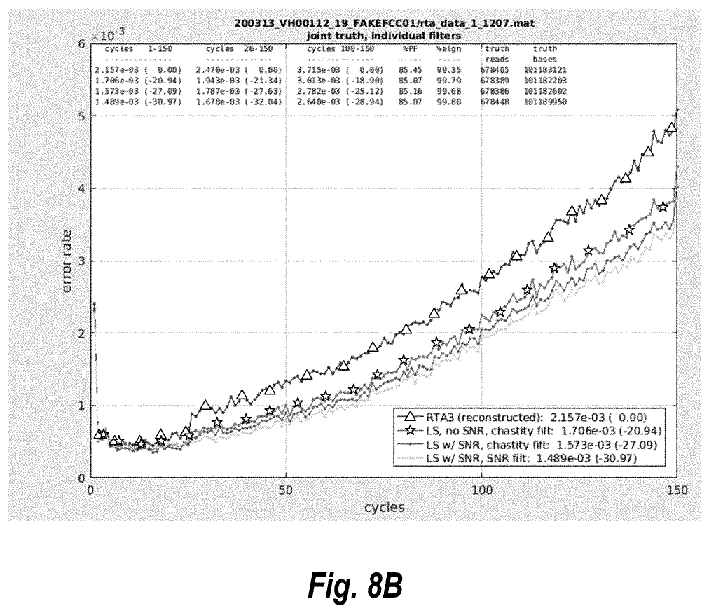

Researchers conducted additional studies to compare the effectiveness of various embodiments of the signal-to-noise-aware base calling system 106. FIGS. 8A-8B illustrate graphs reflecting research results regarding the effectiveness of the signal-to-noise-aware base calling system 106 in accordance with one or more embodiments.

In particular, the graphs of FIGS. 8A-8B compare performance of the embodiments of the signal-to-noise-aware base calling system 106 with a baseline nucleotide-base calling system (labeled “RTAS”). The graphs further compare the performance of one embodiment of the signal-to-noise-aware base calling system 106 that utilizes a chastity filter and does not use distribution-model segmentation (labeled “LS, no SNR, chastity filt”). The graphs show the performance of another embodiment of the signal-to-noise-aware base calling system 106 that uses a chastity filter along with distribution-model segmentation (labeled “LS, w/SNR, chastity filt”). Further, the graphs show performance of yet another embodiment of the signal-to-noise-aware base calling system 106 that uses distribution-model segmentation and filters utilizing the signal-to-noise-ratio threshold (labeled “LS, w/SNR, SNR filt”).

The graph of FIG. 8A illustrates the nucleotide-base call error rate associated with each tested model based on the fraction of sections (e.g., wells) of a nucleotide-sample slide that are analyzed. For example, the fraction of sections analyzed can be based on the fraction of sections that pass the filter implemented by the tested model (e.g., the chastity filter or the filter based on the signal-to-noise-ratio threshold) and that align with a reference (e.g., a reference genome). As shown in FIG. 8A, implementation of the signal-to-noise-ratio metric results in lower nucleotide-base-call error rates. More specifically, use of the distribution-model segmentation and the signal-to-noise-ratio threshold provides the lowest nucleotide-base-call error rates out of all the compared models. As a further note, the graph of FIG. 8A illustrates that adjusting the threshold used to filter out sections of a nucleotide-sample slide has an inverse effect on the error rate (i.e., moving to the right on the x-axis corresponds to a lower threshold and thus a higher percentage of sections that pass the filter, causing a higher error rate).

The graph of FIG. 8B compares the performance of the models across a series of sequencing cycles. As shown, the error rates associated with each model increases as the model progresses through the series of sequencing cycles. The embodiments of the signal-to-noise-aware base calling system 106, however, provide the lowest error rates. Further, as discussed above with reference to the graph of FIG. 8A, use of the distribution-model segmentation and the signal-to-noise-ratio threshold by the signal-to-noise-aware base calling system 106 provides the lowest nucleotide-base-call error rates out of all the compared models. Thus, as shown by both FIG. 8A and FIG. 8B, implementation of the signal-to-noise-ratio metric provides improved accuracy when generating nucleotide-base calls.

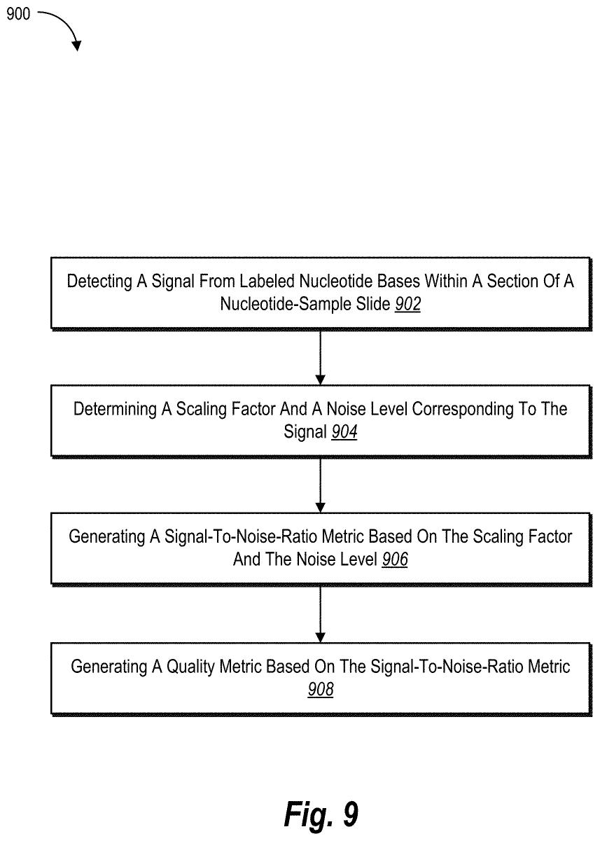

FIGS. 1-8B, the corresponding text and the examples provide a number of different methods, systems, devices, and non-transitory computer-readable media of the signal-to-noise-aware base calling system 106. In addition to the foregoing, one or more embodiments can also be described in terms of flowcharts comprising acts for accomplishing particular results, as shown in FIGS. 9-11. FIGS. 9-11 may be performed with more or fewer acts. Further, the acts may be performed in different orders. Additionally, the acts described herein may be repeated or performed in parallel with one another or in parallel with different instances of the same or similar acts.

FIG. 9 illustrates a flowchart of a series of acts 900 for generating a quality metric for a nucleotide-base call using a signal-to-noise-ratio metric in accordance with one or more embodiments. While FIG. 9 illustrates acts according to one embodiment, alternative embodiments may omit, add to, reorder, and/or modify any of the acts shown in FIG. 9. In some implementations, the acts of FIG. 9 are performed as part of a method. In some instances, a non-transitory computer-readable medium stores instructions thereon that, when executed by at least one processor, cause a computing device to perform the acts of FIG. 9. In some implementations, a system performs the acts of FIG. 9. For example, in one or more cases, a system includes at least one processor and a non-transitory computer readable medium comprising instructions that, when executed by the at least one processor, cause the system to perform the acts of FIG. 9.

The series of acts 900 includes an act 902 for detecting a signal from labeled nucleotide bases within a section of a nucleotide-sample slide. For example, the act 902 can involve detecting a signal from labeled nucleotide bases within a well of a patterned flow cell or a subsection of a non-patterned flow cell.

Additionally, the series of acts 900 includes an act 904 of determining a scaling factor and a noise level corresponding to the signal. For example, the act 904 can involve determining, for the section of the nucleotide-sample slide, a scaling factor and a noise level corresponding to the signal based on intensity values for the signal.

In one or more embodiments, the signal-to-noise-aware base calling system 106 determines, for the section of the nucleotide-sample slide, the noise level corresponding to the signal based on the intensity values for the signal by: determining, for the section of the nucleotide-sample slide, corrected intensity values for the signal; and determining the noise level corresponding to the signal based on the corrected intensity values for the signal. In some cases, the signal-to-noise-aware base calling system 106 determines, for the section of the nucleotide-sample slide, the corrected intensity values for the signal by determining the corrected intensity values based on the intensity values for the signal, the scaling factor corresponding to the signal, and correction offset factors corresponding to the signal. In some instances, the signal-to-noise-aware base calling system 106 determines the noise level corresponding to the signal based on the corrected intensity values for the signal by: determining centroid intensity values for the nucleotide-base call corresponding to the signal; and determining distances between the centroid intensity values and the corrected intensity values for the signal.

In one or more embodiments, the signal-to-noise-aware base calling system 106 determines, for the section of the nucleotide-sample slide, an average noise level for one or more previous sequencing cycles. Accordingly, the signal-to-noise-aware base calling system 106 can determine, for the section for the nucleotide-sample slide, the noise level corresponding to the signal by determining the noise level for a current sequencing cycle based on the average noise level for the one or more previous sequencing cycles.

In some implementations, the signal-to-noise-aware base calling system 106 determines, for the section of the nucleotide-sample slide, a plurality of noise levels for a plurality of previous sequencing cycles; determines a weighted average noise level for the plurality of previous sequencing cycles by applying weighted values to the plurality of noise levels based on sequencing-cycle recency; and determines, for the section for the nucleotide-sample slide, the noise level corresponding to the signal by determining the noise level for a current sequencing cycle based on the weighted average noise level for the plurality of previous sequencing cycles.

In some implementations, the signal-to-noise-aware base calling system 106 determines, for the section of the nucleotide-sample slide, the scaling factor corresponding to the signal based on the intensity values for the signal by: determining a relationship between a measured intensity for the labeled nucleotide bases and variation correction coefficients comprising the scaling factor; determining an error function based on the relationship between the measured intensity and the variation correction coefficients; and determining the scaling factor by generating a partial derivative of the error function with respect to the scaling factor.

Further, the series of acts 900 includes an act 906 of generating a signal-to-noise-ratio metric based on the scaling factor and the noise level. For example, the act 906 can involve generating a signal-to-noise-ratio metric for the section of the nucleotide-sample slide based on the scaling factor and the noise level. In one or more embodiments, the signal-to-noise-aware base calling system 106 generates the signal-to-noise-ratio metric for the section of the nucleotide-sample slide by generating the signal-to-noise-ratio metric for a well of a patterned flow cell or a subsection of a non-patterned flow cell.

The series of acts 900 further includes an act 908 of generating a quality metric based on the signal-to-noise-ratio metric. In particular, the act 908 can involve generating, utilizing a base-call-quality model, a quality metric estimating an error of a nucleotide-base call corresponding to the signal based on the signal-to-noise-ratio metric. In some implementations, the signal-to-noise-aware base calling system 106 generates the quality metric estimating the error of the nucleotide-base call corresponding to the signal based on the signal-to-noise-ratio metric by generating a Phred quality score estimating an accuracy of the nucleotide-base call corresponding to the signal based on the signal-to-noise-ratio metric.

In some implementations, the signal-to-noise-aware base calling system 106 further determines a chastity value for the section of the nucleotide-sample slide based on distances between the intensity values for the signal and intensity values of a nearest centroid and between the intensity values for the signal and intensity values for at least one additional centroid. Accordingly, the signal-to-noise-aware base calling system 106 can generate, utilizing the base-call-quality model, the quality metric based on the signal-to-noise-ratio metric and the chastity value.

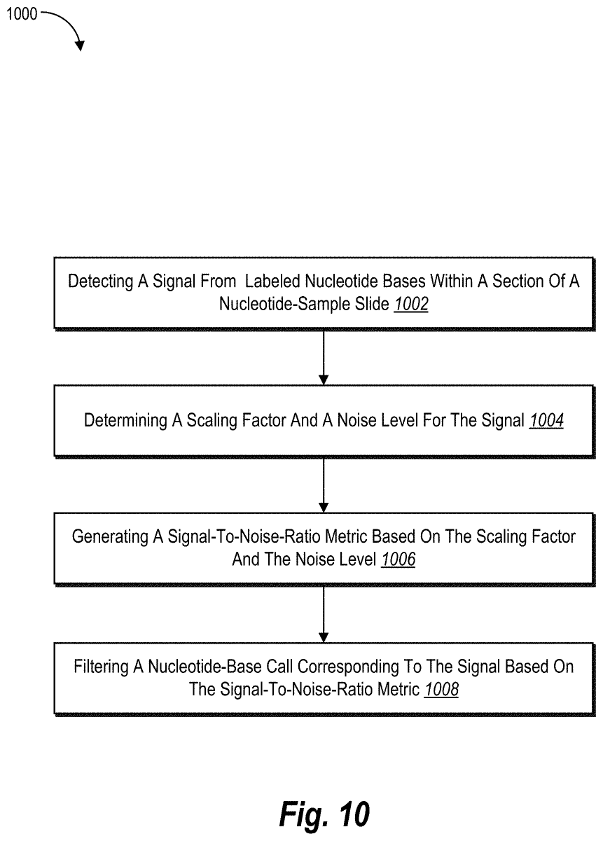

FIG. 10 illustrates a flowchart of a series of acts 1000 for filtering a nucleotide-base call corresponding to a signal using a signal-to-noise-ratio metric in accordance with one or more embodiments. While FIG. 10 illustrates acts according to one embodiment, alternative embodiments may omit, add to, reorder, and/or modify any of the acts shown in FIG. 10. In some implementations, the acts of FIG. 10 are performed as part of a method. In some instances, a non-transitory computer-readable medium stores instructions thereon that, when executed by at least one processor, cause a computing device to perform the acts of FIG. 10. In some implementations, a system performs the acts of FIG. 10. For example, in one or more cases, a system includes at least one processor and a non-transitory computer readable medium comprising instructions that, when executed by the at least one processor, cause the system to perform the acts of FIG. 10.

The series of acts 1000 includes an act 1002 of detecting a signal from labeled nucleotide bases within a section of a nucleotide-sample slide. For example, the act 1002 involves detecting a signal from labeled nucleotide bases within well of a patterned flow cell or a subsection of a non-patterned flow cell. In some instances, the signal-to-noise-aware base calling system 106 detects the signal by detecting the signal from the labeled nucleotide bases incorporated into a growing oligonucleotide at a genomic position later determined in alignment with a reference genome.

The series of acts 1000 also includes an act 1004 of determining a scaling factor and a noise level for the signal. For example, the act 1004 can involve determine, for the section of the nucleotide-sample slide, a scaling factor and a noise level corresponding to the signal based on intensity values for the signal.

In one or more embodiments, the signal-to-noise-aware base calling system 106 determines, for the section of the nucleotide-sample slide, an average noise level for one or more previous sequencing cycles. Accordingly, the signal-to-noise-aware base calling system 106 can determine, for the section for the nucleotide-sample slide, the noise level corresponding to the signal by determining the noise level for a current sequencing cycle based on the average noise level for the one or more previous sequencing cycles.

Additionally, the series of acts 1000 includes an act 1006 of generating a signal-to-noise-ratio metric based on the scaling factor and the noise level. For example, the act 1006 can involve generating a signal-to-noise-ratio metric for the section of the nucleotide-sample slide based on the scaling factor and the noise level. In some instances, the signal-to-noise-aware base calling system 106 generates the signal-to-noise-ratio metric by equating the scaling factor to the signal to determine a ratio of the scaling factor to the noise level. In some cases, the signal-to-noise-aware base calling system 106 generates the signal-to-noise-ratio metric for the nucleotide-base call at the genomic position corresponding to the signal.

Further, the series of acts 1000 includes an act 1008 of filtering a nucleotide-base call corresponding to the signal based on the signal-to-noise-ratio metric. For instance, the act 1008 can involve based on comparing the signal-to-noise-ratio metric to a signal-to-noise-ratio threshold, include or exclude a nucleotide-base call corresponding to the signal within or from nucleotide-base-call data. In some implementations, the signal-to-noise-aware base calling system 106 excludes the nucleotide-base call corresponding to the signal for a well of a patterned flow cell or a subsection of a non-patterned flow cell.

In some implementations, the signal-to-noise-aware base calling system 106 excludes subsequent nucleotide-base calls corresponding to subsequent signals detected from subsequent labeled nucleotide bases added to a cluster of oligonucleotides within the section of the nucleotide-sample slide based on determining that the signal-to-noise-ratio metric is lower than the signal-to-noise-ratio threshold.

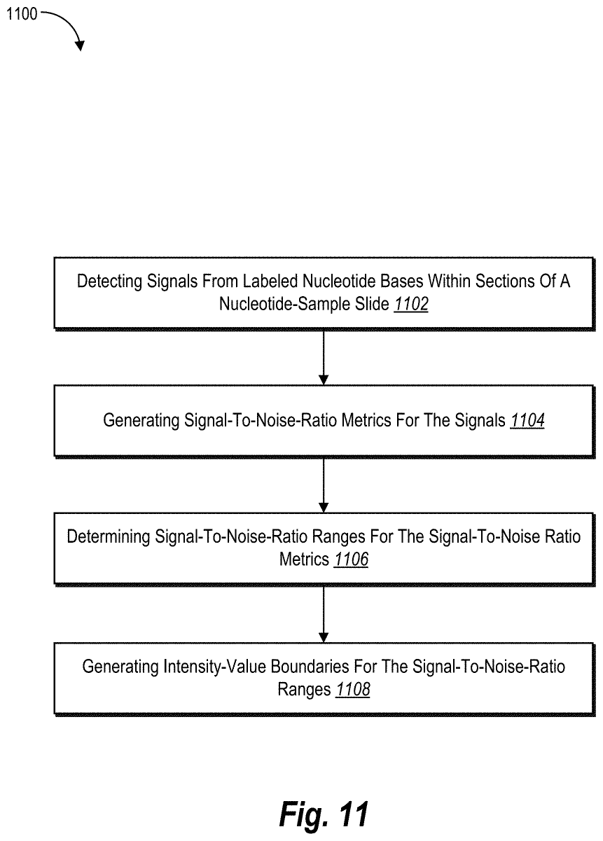

FIG. 11 illustrates a flowchart of a series of acts 1100 for generating intensity-value boundaries for signal-to-noise ranges using signal-to-noise-ratio metrics in accordance with one or more embodiments. While FIG. 11 illustrates acts according to one embodiment, alternative embodiments may omit, add to, reorder, and/or modify any of the acts shown in FIG. 11. In some implementations, the acts of FIG. 11 are performed as part of a method. In some instances, a non-transitory computer-readable medium stores instructions thereon that, when executed by at least one processor, cause a computing device to perform the acts of FIG. 11. In some implementations, a system performs the acts of FIG. 11. For example, in one or more cases, a system includes at least one processor and a non-transitory computer readable medium comprising instructions that, when executed by the at least one processor, cause the system to perform the acts of FIG. 11.

The series of acts 1100 includes an act 1102 of detecting signals from labeled nucleotide bases within sections of a nucleotide-sample slide. For example, the act 1102 can include detecting signals from labeled nucleotide bases within wells of a patterned flow cell or subsections of a non-patterned flow cell.

The series of acts 1100 also includes an act 1104 of generating signal-to-noise-ratio metrics for the signals. For example, the act 1104 can include generating signal-to-noise-ratio metrics for the sections of the at least one nucleotide-sample slide based on the signals and noise levels corresponding to the signals.

The series of acts 1100 further includes an act 1106 of determining signal-to-noise-ratio ranges for the signal-to-noise-ratio metrics. Indeed, the signal-to-noise-aware base calling system 106 can determine a plurality of signal-to-noise-ratio ranges.

Further, the series of acts includes an act 1108 of generating intensity-value boundaries for the signal-to-noise-ratio ranges. For example, the act 1108 can include generating, for each signal-to-noise-ratio range of the signal-to-noise-ratio ranges, intensity-value boundaries for differentiating signals corresponding to different nucleotide bases according to one or more base-call-distribution models. In one or more embodiments, generating the intensity-value boundaries for differentiating the signals corresponding to the different nucleotide bases according to the one or more base-call-distribution models comprises generating the intensity-value boundaries according to on one or more Gaussian distribution models for each signal-to-noise-ratio range of the signal-to-noise-ratio ranges.

In some cases, the signal-to-noise-aware base calling system 106 detects a signal from a subset of labeled nucleotide bases from a cluster of oligonucleotides within a section of a nucleotide-sample slide; generates a signal-to-noise-ratio metric, within a signal-to-noise-ratio range, for the section of the nucleotide-sample slide based on the signal; and determines a nucleotide-base call corresponding to the signal based on a set of intensity-value boundaries of the intensity-value boundaries corresponding to the signal-to-noise-ratio range. Further, the signal-to-noise-aware base calling system 106 can detect an additional signal from an additional subset of labeled nucleotide bases from an additional cluster of oligonucleotides within an additional section of the nucleotide-sample slide; generate an additional signal-to-noise-ratio metric, within an additional signal-to-noise-ratio range, for the additional section of the nucleotide-sample slide based on the additional signal, wherein the additional signal-to-noise-ratio range differs from the signal-to-noise-ratio range; and determine an additional nucleotide-base call corresponding to the additional signal based on an additional set of intensity-value boundaries of the intensity-value boundaries corresponding to the additional signal-to-noise-ratio range.

In one or more embodiments, generating, for each signal-to-noise-ratio range of the signal-to-noise-ratio ranges, the intensity-value boundaries for differentiating the signals corresponding to the different nucleotide bases according to the one or more base-call-distribution models comprises: generating, for a first signal-to-noise-ratio range, a first set of intensity-value boundaries corresponding to the different nucleotide bases according to a first base-call-distribution model; and generating, for a second signal-to-noise-ratio range, a second set of intensity-value boundaries corresponding to the different nucleotide bases according to a second base-call-distribution model, the second set of intensity-value boundaries differing from the first set of intensity-value boundaries.

In some cases, the signal-to-noise-aware base calling system 106 detects a first signal corresponding to a first signal-to-noise-ratio metric within the first signal-to-noise-ratio range and having a set of intensity values outside of the first set of intensity-value boundaries and outside the second set of intensity-value boundaries; detects a second signal corresponding to a second signal-to-noise-ratio metric within the second signal-to-noise-ratio range and having the set of intensity values; generates a first nucleotide-base call for the first signal based on the first set of intensity-value boundaries for the first base-call-distribution model; and generates a second nucleotide-base call for the second signal based on the second set of intensity-value boundaries for the second base-call-distribution model.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the signal-to-noise-aware base calling system 106 can include software, hardware, or both. For example, the components of the signal-to-noise-aware base calling system 106 can include one or more instructions stored on a computer-readable storage medium and executable by processors of one or more computing devices. When executed by the one or more processors, the computer-executable instructions of the signal-to-noise-aware base calling system 106 can cause the computing devices to perform the bubble detection methods described herein. Alternatively, the components of the signal-to-noise-aware base calling system 106 can comprise hardware, such as special purpose processing devices to perform a certain function or group of functions. Additionally, or alternatively, the components of the signal-to-noise-aware base calling system 106 can include a combination of computer-executable instructions and hardware.

Furthermore, the components of the signal-to-noise-aware base calling system 106 performing the functions described herein with respect to the signal-to-noise-aware base calling system 106 may, for example, be implemented as part of a stand-alone application, as a module of an application, as a plug-in for applications, as a library function or functions that may be called by other applications, and/or as a cloud-computing model. Thus, components of the signal-to-noise-aware base calling system 106 may be implemented as part of a stand-alone application on a personal computing device or a mobile device. Additionally, or alternatively, the components of the signal-to-noise-aware base calling system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 12 illustrates a block diagram of a computing device 1200 that may be configured to perform one or more of the processes described above. One will appreciate that one or more computing devices such as the computing device 1200 may implement the signal-to-noise-aware base calling system 106 and the sequencing system 104. As shown by FIG. 12, the computing device 1200 can comprise a processor 1202, a memory 1204, a storage device 1206, an I/O interface 1208, and a communication interface 1210, which may be communicatively coupled by way of a communication infrastructure 1212. In certain embodiments, the computing device 1200 can include fewer or more components than those shown in FIG. 12. The following paragraphs describe components of the computing device 1200 shown in FIG. 12 in additional detail.

In one or more embodiments, the processor 1202 includes hardware for executing instructions, such as those making up a computer program. As an example, and not by way of limitation, to execute instructions for dynamically modifying workflows, the processor 1202 may retrieve (or fetch) the instructions from an internal register, an internal cache, the memory 1204, or the storage device 1206 and decode and execute them. The memory 1204 may be a volatile or non-volatile memory used for storing data, metadata, and programs for execution by the processor(s). The storage device 1206 includes storage, such as a hard disk, flash disk drive, or other digital storage device, for storing data or instructions for performing the methods described herein.

The I/O interface 1208 allows a user to provide input to, receive output from, and otherwise transfer data to and receive data from computing device 1200. The I/O interface 1208 may include a mouse, a keypad or a keyboard, a touch screen, a camera, an optical scanner, network interface, modem, other known I/O devices or a combination of such I/O interfaces. The I/O interface 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1208 is configured to provide graphical data to a display for presentation to a user. The graphical data may be representative of one or more graphical user interfaces and/or any other graphical content as may serve a particular implementation.

The communication interface 1210 can include hardware, software, or both. In any event, the communication interface 1210 can provide one or more interfaces for communication (such as, for example, packet-based communication) between the computing device 1200 and one or more other computing devices or networks. As an example, and not by way of limitation, the communication interface 1210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1210 may facilitate communications with various types of wired or wireless networks. The communication interface 1210 may also facilitate communications using various communication protocols. The communication infrastructure 1212 may also include hardware, software, or both that couples components of the computing device 1200 to each other. For example, the communication interface 1210 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
detect a signal from labeled nucleotide bases within a section of a nucleotide-sample slide;
determine, for the section of the nucleotide-sample slide, a scaling factor and a noise level corresponding to the signal based on intensity values for the signal;
generate a signal-to-noise-ratio metric for the section of the nucleotide-sample slide based on the scaling factor and the noise level; and
generate, utilizing a base-call-quality model, a quality metric estimating an error of a nucleotide-base call corresponding to the signal based on the signal-to-noise-ratio metric.

2. The system of claim 1, further comprising instructions that, when executed by the at least one processor, cause the system to determine, for the section of the nucleotide-sample slide, the noise level corresponding to the signal based on the intensity values for the signal by:
determining, for the section of the nucleotide-sample slide, corrected intensity values for the signal; and
determining the noise level corresponding to the signal based on the corrected intensity values for the signal.

3. The system of claim 2, further comprising instructions that, when executed by the at least one processor, cause the system to determine, for the section of the nucleotide-sample slide, the corrected intensity values for the signal by determining the corrected intensity values based on the intensity values for the signal, the scaling factor corresponding to the signal, and correction offset factors corresponding to the signal.

4. The system of claim 2, further comprising instructions that, when executed by the at least one processor, cause the system to determine the noise level corresponding to the signal based on the corrected intensity values for the signal by:
determining centroid intensity values for the nucleotide-base call corresponding to the signal; and
determining distances between the centroid intensity values and the corrected intensity values for the signal.

5. The system of claim 1, further comprising instructions that, when executed by the at least one processor, cause the system to:
determine, for the section of the nucleotide-sample slide, an average noise level for one or more previous sequencing cycles; and
determine, for the section for the nucleotide-sample slide, the noise level corresponding to the signal by determining the noise level for a current sequencing cycle based on the average noise level for the one or more previous sequencing cycles.

6. The system of claim 1, further comprising instructions that, when executed by the at least one processor, cause the system to determine, for the section of the nucleotide-sample slide, the scaling factor corresponding to the signal based on the intensity values for the signal by:
determining a relationship between a measured intensity for the labeled nucleotide bases and variation correction coefficients comprising the scaling factor;
determining an error function based on the relationship between the measured intensity and the variation correction coefficients; and
determining the scaling factor by generating a partial derivative of the error function with respect to the scaling factor.

7. The system of claim 1, further comprising instructions that, when executed by the at least one processor, cause the system to generate the signal-to-noise-ratio metric for the section of the nucleotide-sample slide by generating the signal-to-noise-ratio metric for a well of a patterned flow cell or a subsection of a non-patterned flow cell.

8. The system of claim 1, further comprising instructions that, when executed by the at least one processor, cause the system to generate the quality metric estimating the error of the nucleotide-base call corresponding to the signal based on the signal-to-noise-ratio metric by generating a Phred quality score estimating an accuracy of the nucleotide-base call corresponding to the signal based on the signal-to-noise-ratio metric.

9. The system of claim 1, further comprising instructions that, when executed by the at least one processor, cause the system to:
determine a chastity value for the section of the nucleotide-sample slide based on distances between the intensity values for signal and intensity values of a nearest centroid and between the intensity values for the signal and intensity values for at least one additional centroid; and
generate, utilizing the base-call-quality model, the quality metric based on the signal-to-noise-ratio metric and the chastity value.

10. The system of claim 1, further comprising instructions that, when executed by the at least one processor, cause the system to:
determine, for the section of the nucleotide-sample slide, a plurality of noise levels for a plurality of previous sequencing cycles;
determine a weighted average noise level for the plurality of previous sequencing cycles by applying weighted values to the plurality of noise levels based on sequencing-cycle recency; and
determine, for the section for the nucleotide-sample slide, the noise level corresponding to the signal by determining the noise level for a current sequencing cycle based on the weighted average noise level for the plurality of previous sequencing cycles.

11. A non-transitory computer-readable medium storing instructions thereon that, when executed by at least one processor, cause a computing device to:
detect a signal from labeled nucleotide bases within a section of a nucleotide-sample slide;
determine, for the section of the nucleotide-sample slide, a scaling factor and a noise level corresponding to the signal based on intensity values for the signal;
generate a signal-to-noise-ratio metric for the section of the nucleotide-sample slide based on the scaling factor and the noise level; and
based on comparing the signal-to-noise-ratio metric to a signal-to-noise-ratio threshold, include or exclude a nucleotide-base call corresponding to the signal within or from nucleotide-base-call data.

12. The non-transitory computer-readable medium of claim 11, further comprising instructions that, when executed by the at least one processor, cause the computing device to exclude subsequent nucleotide-base calls corresponding to subsequent signals detected from subsequent labeled nucleotide bases added to a cluster of oligonucleotides within the section of the nucleotide-sample slide based on determining that the signal-to-noise-ratio metric is lower than the signal-to-noise-ratio threshold.

13. The non-transitory computer-readable medium of claim 11, further comprising instructions that, when executed by the at least one processor, cause the computing device to generate the signal-to-noise-ratio metric by equating the scaling factor to the signal to determine a ratio of the scaling factor to the noise level.

14. The non-transitory computer-readable medium of claim 11, further comprising instructions that, when executed by the at least one processor, cause the computing device to:
detect the signal by detecting the signal from the labeled nucleotide bases incorporated into a growing oligonucleotide at a genomic position later determined in alignment with a reference genome; and
generate the signal-to-noise-ratio metric for the nucleotide-base call at the genomic position corresponding to the signal.

15. A method comprising:
detecting signals from labeled nucleotide bases within sections of at least one nucleotide-sample slide;
generating signal-to-noise-ratio metrics for the sections of the at least one nucleotide-sample slide based on the signals and noise levels corresponding to the signals;
determining signal-to-noise-ratio ranges for the signal-to-noise-ratio metrics; and
generating, for each signal-to-noise-ratio range of the signal-to-noise-ratio ranges, intensity-value boundaries for differentiating signals corresponding to different nucleotide bases according to one or more base-call-distribution models.

16. The method of claim 15, wherein generating, for each signal-to-noise-ratio range of the signal-to-noise-ratio ranges, the intensity-value boundaries for differentiating the signals corresponding to the different nucleotide bases according to the one or more base-call-distribution models comprises:
generating, for a first signal-to-noise-ratio range, a first set of intensity-value boundaries corresponding to the different nucleotide bases according to a first base-call-distribution model; and
generating, for a second signal-to-noise-ratio range, a second set of intensity-value boundaries corresponding to the different nucleotide bases according to a second base-call-distribution model, the second set of intensity-value boundaries differing from the first set of intensity-value boundaries.

17. The method of claim 16, further comprising:
detecting a first signal corresponding to a first signal-to-noise-ratio metric within the first signal-to-noise-ratio range and having a set of intensity values outside of the first set of intensity-value boundaries and outside the second set of intensity-value boundaries;
detecting a second signal corresponding to a second signal-to-noise-ratio metric within the second signal-to-noise-ratio range and having the set of intensity values;
generating a first nucleotide-base call for the first signal based on the first set of intensity-value boundaries for the first base-call-distribution model; and
generating a second nucleotide-base call for the second signal based on the second set of intensity-value boundaries for the second base-call-distribution model.

18. The method of claim 15, further comprising:
detecting a signal from a subset of labeled nucleotide bases from a cluster of oligonucleotides within a section of a nucleotide-sample slide;
generating a signal-to-noise-ratio metric, within a signal-to-noise-ratio range, for the section of the nucleotide-sample slide based on the signal; and
determining a nucleotide-base call corresponding to the signal based on a set of intensity-value boundaries of the intensity-value boundaries corresponding to the signal-to-noise-ratio range.

19. The method of claim 18, further comprising:
detecting an additional signal from an additional subset of labeled nucleotide bases from an additional cluster of oligonucleotides within an additional section of the nucleotide-sample slide;
generating an additional signal-to-noise-ratio metric, within an additional signal-to-noise-ratio range, for the additional section of the nucleotide-sample slide based on the additional signal, wherein the additional signal-to-noise-ratio range differs from the signal-to-noise-ratio range; and
determining an additional nucleotide-base call corresponding to the additional signal based on an additional set of intensity-value boundaries of the intensity-value boundaries corresponding to the additional signal-to-noise-ratio range.

20. The method of claim 15, wherein generating the intensity-value boundaries for differentiating the signals corresponding to the different nucleotide bases according to the one or more base-call-distribution models comprises generating the intensity-value boundaries according to on one or more Gaussian distribution models for each signal-to-noise-ratio range of the signal-to-noise-ratio ranges.
